(19) United States
(12) Patent Application Publication
(10) Pub. No.: US 2023/0160864 A1
(43) Pub. Date: May 25, 2023

(54) METHODS OF MEASURING AND PURIFYING EXTRACELLULAR VESICLES

(71) Applicants: President and Fellows of Harvard College, Cambridge, MA (US); The Brigham and Women's Hospital, Inc., Boston, MA (US)

(72) Inventors: David R. Walt, Boston, MA (US); Dmitry Ter-Ovanesyan, Boston, MA (US); Maia Norman, Cambridge, MA (US)

(21) Appl. No.: 17/798,937

(22) PCT Filed: Feb. 12, 2021
(86) PCT No.: PCT/US21/17770

§ 371 Date: Aug. 11, 2022

Related U.S. Application Data
(60) Provisional application No. 62/975,506, filed on Feb. 12, 2020.

(51) Int. Cl. G01N 30/88; B01J 39/26; G01N 33/564; G01N 33/566

(52) U.S. Cl. CPC G01N 30/88; B01J 39/26; G01N 33/564; G01N 33/566

(57) ABSTRACT

The present disclosure provides novel methods to purify and quantify extracellular vesicles (EVs) in biological samples, e.g plasma or cerebrospinal fluid (CSF).

### CROSS-REFERENCE TO RELATED APPLICATIONS

The present application claims priority to U.S. Provisional Application No. 62/975,506, filed on Feb. 12, 2020, the entire contents of which are incorporated herein by reference.

### BACKGROUND OF THE DISCLOSURE

Extracellular vesicles (EVs) are released by all cell types, and are found in biological fluids such as plasma and CSF. As EVs contain RNA and protein from their donor cells, EVs provide a broad platform for non-invasively obtaining molecular information about cell types in the human body that are inaccessible to biopsy and, thus, hold great promise as diagnostic biomarkers.

Despite the diagnostic potential of EVs, there are several challenges that have hampered their utility as biomarkers. First, EVs are heterogeneous and difficult to quantify. Additionally, EVs and their contents are present at low amounts in clinically relevant biosamples where volumes are limited. Finally, and partly due to a lack of suitable quantification methods, there is a lack of consensus about the best way of purifying EVs from plasma and other biofluids.

Several methods have been used in attempts to quantitate EVs. These methods, such as nanoparticle tracking analysis (NTA), dynamic light scattering (DLS), and tunable resistive pulse sensing (TRPS), aim to measure both particle size and concentration. A major limitation of these methods is that they cannot discriminate lipoproteins or particles of aggregated proteins from EVs. Since biofluids, and plasma in particular, contain an abundance of lipoproteins and protein aggregates at levels higher than those of EVs, these methods are ill-suited for quantitating EVs. As the existence of a membrane is a defining characteristic of EVs, lipid dyes have also been used to measure EVs. These dyes also bind to lipoproteins, however, and lack sensitivity.

Accordingly, there remains a need in the art for a highly sensitive and efficient method for purifying and quantifying EVs from various biological fluids.

### SUMMARY OF THE DISCLOSURE

The present disclosure provides novel methods to purify and quantify extracellular vesicles (EVs) in biological samples, e.g., plasma or cerebrospinal fluid (CSF).

In some aspects, the current disclosure provides a method for purifying extracellular vesicles from a biological sample, the method comprising: combining a liquid biological sample containing extracellular vesicles with size-exclusion beads capable of capturing molecules smaller than about 700 kDa to create a mixture, and separating and removing the size-exclusion beads from the mixture, such that the extracellular vesicles remain, thereby purifying the extracellular vesicles.

In some embodiments, the biological sample is obtained from a subject. In some embodiments, the liquid biological sample is a plasma sample. In some embodiments, the liquid biological sample is a cerebrospinal fluid (CSF) sample.

In some embodiments, the size-exclusion beads are suspended in a buffer to create a slurry. In some embodiments, the size-exclusion beads are suspended in an equal volume of buffer to create a slurry. In some embodiments, the buffer is PBS buffer.

In some embodiments, the mixture is mixed prior to separating and removing the size-exclusion beads. In some embodiments, the mixture is mixed for between 30 minutes and 1 hour. In some embodiments, the size-exclusion beads are separated from the mixture using centrifugation. In some embodiments, the centrifugation is at about 800 g.

In some embodiments, following separating and removing the size-exclusion beads from the mixture, the remaining extracellular vesicles are further purified.

In some embodiments, the size-exclusion beads comprise an inactive bead exterior. In some embodiments, the interior of the beads comprise octylamine ligands. In some embodiments, the size-exclusion beads comprise bind-elute resin. In some embodiments, the size-exclusion beads are Capto™ Core 700 bind-elute beads.

In some embodiments, the size-exclusion beads are added in an amount of 20 μL, 50 μL, 100 μL, or 200 μL per 1 mL of liquid biological sample.

In some embodiments, the biological sample is subjected to a size exclusion chromatography (SEC) column prior to combining with the size-exclusion beads capable of capturing molecules smaller than about 700 kDa. In some embodiments, the size exclusion chromatography column comprises a stationary phase comprising a 6% cross-linked agarose size exclusion chromatography base matrix. In some embodiments, the 6% cross-linked agarose size exclusion chromatography base matrix is a Sepharose™ CL-6B resin.

In some embodiments, the biological sample is further subjected to a cation exchange chromatography resin after the size exclusion chromatography column and prior to combining with the size-exclusion beads capable of capturing molecules smaller than about 700 kDa. In some embodiment, the cation exchange chromatography resin is Fractogel® EMD-SO₃⁻ resin.

In some embodiments, the method is a high-throughput method.

In other aspects, the current disclosure provides a method for purifying extracellular vesicles from a biological sample, the method comprising: providing a size exclusion chromatography (SEC) column comprising a stationary phase comprising a 6% cross-linked agarose size exclusion chromatography base matrix, introducing a liquid biological sample comprising extracellular vesicles into the column, and collecting fractions containing extracellular vesicles from the column, thereby purifying the extracellular vesicles.

In some embodiments, the 6% cross-linked agarose size exclusion chromatography base matrix is a Sepharose™ CL-6B resin.

In some embodiments, the liquid biological sample is obtained from a subject. In some embodiments, the liquid biological sample is a plasma sample. In some embodiments, the liquid biological sample is a cerebrospinal fluid (CSF) sample.

In some embodiments, the SEC column is a 10 mL volume column. In some embodiments, the SEC column is a 20 mL volume column.

In some embodiments, fractions 6-21 are collected from the column. In some embodiments, fractions 12-27 are collected from the column.

In some embodiments, the 6% cross-linked agarose size exclusion chromatography base matrix is washed in buffer prior to preparation of the column.

In some embodiments, the 6% cross-linked agarose size exclusion chromatography base matrix is washed multiple times in buffer prior to preparation of the column. In some embodiments, the 6% cross-linked agarose size exclusion chromatography base matrix is washed four or more times in buffer prior to preparation of the column. In some embodiments, the buffer is PBS buffer.

In some embodiments, the column comprising the 6% cross-linked agarose size exclusion chromatography base matrix is washed prior to contacting it with the sample. In some embodiments, the column is washed with PBS prior to contacting it with the sample.

In some embodiments, the fractions collected from the SEC column are further subjected to a cation exchange chromatography resin. In some embodiments, the cation exchange chromatography resin comprises a stationary phase comprising a functional group selected from the group consisting of sulpfhydryl, sulfonate, sulfate, carboxymethyl, sulfoethyl, sulfopropyl, phosphate and sulfonate. In some embodiments, the cation exchange chromatography resin is Fractogel® EMD-SO₃⁻ resin.

In some embodiments, the fractions collected from the SEC column are further subjected to a size-exclusion beads capable of capturing molecules smaller than about 700 kDa. In some embodiments, the size-exclusion beads are Capto™ Core 700.

In some embodiments, the fractions collected from the SEC column are further subjected to a cation exchange chromatography resin and a size-exclusion chromatography capable of capturing molecules smaller than about 700 kDa. In some embodiments, the cation exchange chromatography resin is Fractogel® EMD-SO₃⁻ resin. In some embodiments, the size-exclusion beads are Capto™ Core 700.

In other aspects, the current disclosure provides a method for detecting a single antigen-antibody immunocomplex of an antigen in a biological sample containing the antigen-antibody immunocomplex, wherein the antigen is a tetraspanin from an extracellular vesicle (EV), the method comprising (a) contacting a biological sample containing the antigen-antibody immunocomplex with a plurality of capture objects, wherein each capture object comprises a capture probe coupled to a plurality of capture moieties that specifically bind to the antigen-antibody immunocomplex, wherein the biological sample and the plurality of capture objects are contacted at a ratio that allows for a single antigen-antibody immunocomplex to bind to a single capture object; (b) incubating the product of step (a) for a sufficient time to allow binding of the plurality of capture objects to the antigen-antibody immunocomplex contained in the sample, thereby creating a complex of the capture object and the antigen-antibody immunocomplex; (c) contacting the complex of the capture object and the antigen-antibody immunocomplex from step (b) with a plurality of detection probes, wherein the detection probes bind to the antigen-antibody immunocomplex within the complex of the capture object and the antigen-antibody immunocomplex from step (b); (d) labeling the product of step (c) with a detectable moiety; and (e) detecting the detectable moiety, thereby detecting the single antigen-antibody immunocomplex in the sample, wherein the antigen is a tetraspanin comprised in an extracellular vesicle (EV).

In some embodiments, the tetraspanin antigen is selected from the group consisting of CD9, CD63, and CD81. In other embodiments, the tetraspanin antigen is selected from those listed in Table 1.

In some embodiments, the biological sample is a liquid biological sample. In some embodiments, the biological sample is a plasma sample. In some embodiments, the biological sample is a cerebrospinal fluid (CSF) sample.

In some embodiments, the method further comprises quantifying the antigen-antibody immunocomplex in the sample by determining the number of antigen-antibody immunocomplexes bound to the capture objects.

In some embodiments, the capture moiety on the capture probe is a full-length antibody, an antigen-binding fragment of an antibody, or an antibody mimetic.

In some embodiments, step (e) comprises linking a detection probe to the detectable moiety by a non-covalent affinity binding pair, wherein the detection probe is linked to a first member of the non-covalent affinity binding pair, and the detectable moiety is linked to a second member of the non-covalent affinity binding pair.

In some embodiments, the detection of step (e) comprises single-molecule detection of the detectable moiety.

In some embodiments, the detection of step (e) occurs in an array of microwells, wherein the microwells are capable of holding zero or one capture objects.

In some embodiments, the detectable moiety is detected in a fluid volume of about 10 attoliters (aL) to about 10 picoliters (pL) in a microwell.

In some embodiments, the array is a QUANTERIX™ single molecule array (Simoa).

In some embodiments, the microwells have a volume of about 40 femtoliters (fL).

In some embodiments, the method further comprises quantifying the purified extracellular vesicles (EVs) using the one or more of the methods of the disclosure.

In other aspects, the current disclosure provides a method for quantifying extracellular vesicles in a biological sample comprising detecting a single antigen-antibody immunocomplex of an antigen in a biological sample containing the antigen-antibody immunocomplex, wherein the antigen is a tetraspanin from an extracellular vesicle (EV), the method comprising: (a) contacting a biological sample containing the antigen-antibody immunocomplex with a plurality of capture objects, wherein each capture object comprises a capture probe coupled to a plurality of capture moieties that specifically bind to the antigen-antibody immunocomplex, wherein the biological sample and the plurality of capture objects are contacted at a ratio that allows for a single antigen-antibody immunocomplex to bind to a single capture object; (b) incubating the product of step (a) for a sufficient time to allow binding of the plurality of capture objects to the antigen-antibody immunocomplex contained in the sample, thereby creating a complex of the capture object and the antigen-antibody immunocomplex; (c) contacting the complex of the capture object and the antigen-antibody immunocomplex from step (b) with a plurality of detection probes, wherein the detection probes bind to the antigen-antibody immunocomplex within the complex of the capture object and the antigen-antibody immunocomplex from step (b); (d) labeling the product of step (c) with a detectable moiety; and (e) detecting the detectable moiety, thereby detecting the single antigen-antibody immunocomplex in the sample, and detecting the EVs in the sample.

Additional features and advantages of the disclosure will be apparent from the following detailed descriptions, the drawings, and the claims.

### BRIEF DESCRIPTION OF THE DRAWINGS

FIGS. 1A-D represent a schematic illustration of an exemplary QUANTERIX™ single molecule array (Simoa®) assay method of the disclosure. A. Plasma or CSF were cleared of cells and cellular debris by centrifugation and filtration with a 0.45 UL filter. B. Plasma or CSF were fractionated by size exclusion chromatography utilizing a variety of resins. C. Fractions were measured using single molecule array assays. D. Schematic representation of distribution of extracellular vesicles and free proteins in fractionated biological fluid.

FIGS. 2A-F depict a comparison of calibration curves obtained from ELISA and QUANTERIX™ single molecule array (Simoa®) assays for EV quantification. Simoa® was able to quantify EV fractions 7-10 for all three transmembrane proteins in both cerebrospinal fluid (CSF) and plasma. A. CD9 ELISA calibration curve (left) LOD 99.7 pM, concentration in CSF after fractionation with an Izon 35 nM column (middle), concentration in plasma after fractionation with an Izon 35 nM column (right). Error bars represent two assay replicates. B. CD9 Simoa calibration curve (left) LOD 0.231 pM, concentration in CSF after fractionation with an Izon 35 nM column (middle), concentration in plasma after fractionation with an Izon 35 nM column (right). Error bars represent two assay replicates. C. CD63 ELISA calibration curve (left) 0.142 pM, concentration in CSF after fractionation with an Izon 35 nM column (middle), concentration in plasma after fractionation with an Izon 35 nM column (right). Error bars represent two assay replicates. D. CD63 Simoa calibration curve (left) 0.0121 pM, concentration in CSF after fractionation with an Izon 35 nM column (middle), concentration in plasma after fractionation with an Izon 35 nM column (right). Error bars represent two assay replicates. E. CD81 ELISA calibration curve (left) 33.1 pM, concentration in CSF after fractionation with an Izon 35 nM column (middle), concentration in plasma after fractionation with an Izon 35 nM column (right). Error bars represent two assay replicates. F. CD81 Simoa calibration curve (left) 0.697 pM, concentration in CSF after fractionation with an Izon 35 nM column (middle), concentration in plasma after fractionation with an Izon 35 nM column (right). Error bars represent two assay replicates.

FIGS. 3A-B are graphs showing a comparison of commercial methods for EV purification in CSF (FIG. 3A) and plasma (FIG. 3B) for each of transmembrane proteins CD9, CD63, and CD81 and albumin. Comparison of commercial methods for EV purification in plasma and CSF. A. Comparison of commercial purification in plasma: Individual tetraspanin yield (top), relative EV recovery was calculated by first normalizing individual tetraspanin values (in pM) in each technique to that of Izon35 nM F7-10 and then averaging the three tetraspanins percentages (middle) and albumin contamination (bottom) in order of relative albumin contamination. B. Comparison of commercial purification in plasma: Individual tetraspanin yield (top), relative EV recovery was calculated by first normalizing individual tetraspanin values (in pM) in each technique to that of Izon35 nM F7-10 and then averaging the three tetraspanins percentages (middle) and albumin contamination (bottom) in order of relative albumin contamination.

FIGS. 4A-C depict the results of comparison of size exclusion chromatography (SEC) methods for EV purification in plasma for each of transmembrane proteins CD9, CD63, and CD81 and albumin, using a Sepharose™ 10 mL column, an Izon column, and a Sepharose™ 20 mL column. A. Concentration of EV markers and albumin in plasma after fractionation with 10 mL homebrew columns; Sepharose™ CL-6B (top), Sepharose™ CL-4B (middle), Sepharose™ CL-2B (bottom). Error bars represent two assay replicates. B. Concentration of EV markers and albumin in plasma after fractionation with Izon 35 nM column (top) and Izon 70 nM column (bottom). Error bars represent two assay replicates. C. Concentration of EV markers and albumin in plasma after fractionation with 20 mL homebrew columns; Sepharose™ CL-6B (top), Sepharose™ CL-4B (middle), Sepharose™ CL-2B (bottom). Error bars represent two assay replicates.

FIG. 5A-C depict the results of comparison of size exclusion chromatography (SEC) methods for EV purification in CSF for each of transmembrane proteins CD9, CD63, and CD81 and albumin using a Sepharose™ 10 mL column, an Izon column, and a Sepharose™ 20 mL column. A. Concentration of EV markers and albumin in CSF after fractionation with 10 mL homebrew columns; Sepharose™ CL-6B (top), Sepharose™ CL-4B (middle), Sepharose™ CL-2B (bottom). Error bars represent two assay replicates. B. Concentration of EV markers and albumin in CSF after fractionation with Izon 35 nM column (top) and Izon 70 nM column (bottom). Error bars represent two assay replicates. C. Concentration of EV markers and albumin in CSF after fractionation with 20 mL homebrew columns; Sepharose™ CL-6B (top), Sepharose™ CL-4B (middle), Sepharose™ CL-2B (bottom). Error bars represent two assay replicates.

FIG. 6A depicts a comparison of plasma EV recovery (top) and albumin contamination (bottom) across all tested methods ranked by EV recovery. Everything was normalized to the sum of all fractions in the Sepharose™ CL-6B 10 mL condition.

FIG. 6B depicts a comparison of CSF EV recovery (top) and albumin contamination (bottom) across all tested methods ranked by EV recovery. Everything was normalized to the sum of all fractions in the Sepharose™ CL-6B 10 mL condition.

FIG. 7A-D depicts endogenous dilution linearity of Simoa Assays in CSF and Plasma. A. Endogenous dilution linearity (parallelism) of CD9 in 4 samples of CSF (top) and plasma (bottom). B. Endogenous dilution linearity (parallelism) of CD63 in 4 samples of CSF (top) and plasma (bottom). C. Endogenous dilution linearity (parallelism) of CD81 in 4 samples of CSF (top) and plasma (bottom). D. Endogenous dilution linearity (parallelism) of Albumin in 4 samples of CSF (top) and plasma (bottom).

FIG. 8 is a Western blot analysis of SEC fractions loaded with equal volume of each fraction (not normalized for protein quantity).

FIG. 9A shows graphs depicting effect of volume on EV recovery and purity for transmembrane proteins CD9, CD63 and CD81, and albumin across 0.1 mL (top), 0.5 mL (middle) and 1.0 mL (bottom) of CSF.

FIG. 9B shows graphs depicting the effect of volume on EV recovery and purity for transmembrane proteins CD9, CD63 and CD81, and albumin across 0.1 mL (top), 0.5 mL (middle) and 1.0 mL (bottom) of plasma.

FIGS. 10A-C depict purification of EV from human CSF samples using Capto Core 700 beads. FIG. 10A depicts a Western blot analysis for the transmembrane proteins CD9, CD63 and CD81 after increasing the amounts of Capto Core 700 beads (1 Captor Core 700 per 10 μg, 5 μg or 2.5 μg of total protein) (left) and a bar graph representing the percentage recovery for each protein (right). FIG. 10B depicts a Western blot analysis for the contaminating protein albumin after increasing the amounts of Capto Core 700 beads (1 μL Captor Core 700 per 10 μs, 5 μg or 2.5 μg of total protein) (left) and a bar graph representing the percentage recovery of albumin (right). FIG. 10C depicts the total protein stain after increasing the amounts of Capto Core 700 beads (1 μL Captor Core 700 per 10 μg, 5 μg or 2.5 μg of total protein).

FIGS. 11A-B depict purification of EV from human plasma samples using size exclusion chromatography with a 10 mL Sepharose™ CL-6B column followed by Capto Core 700 beads. Fractions 7-10 were collected from SEC and added with various amount of Capto Core 700 beads. FIG. 11A depicts a Western blot analysis for the transmembrane proteins CD9, CD63 and CD81 and the contaminating protein albumin after increasing the amounts of Capto Core 700 beads (1 μL Captor Core 700 per 2.5 μg, 1.25 μg or 0.6 μs of total protein) (left) and a bar graph representing the percentage recovery for each protein (right). FIG. 11B depicts the total protein stain after increasing the amounts of Capto Core 700 beads (1 μL Captor Core 700 per 2.5 μg, 1.25 μg or 0.6 μs of total protein) added to fractions 7-10 of plasma SEC.

FIG. 12A-B depict a comparison between fractionation of human plasma samples by size exclusion chromatography (SEC) with a 10 mL Sepharose™ CL-6B column or dual mode chromatography (DMC) using 10 mL Sepharose™ CL-6B layered on top of 2 mL of cation exchange resin (Fractogel EMD SO₃⁻). FIG. 12A depicts a Western blot analysis of a major lipoprotein component ApoB 100 (top) and an EV marker CD81 (bottom). FIG. 12B depicts a bar graph representing the percentage recovery of ApoB100 (top) and CD81 (bottom) after DMC relative to SEC.

FIG. 13 depict a comparison between fractionation of human plasma samples by (1) size exclusion chromatography (SEC) with a 10 mL Sepharose™ CL-6B column, (2) SEC followed by CaptoCore 700 beads, (3) dual mode chromatography (DMC) using 10 mL Sepharose™ CL-6B layered on top of 2 mL of cation exchange resin (Fractogel EMD SO₃⁻), and (4) DMC followed by Capto Core 700 beads. Specifically, FIG. 13 depicts a Western blot analysis of a major lipoprotein component ApoB 100 (top), a contaminating protein Albumin (middle), and a EV marker CD81 (bottom).

FIG. 14 is a schematic representation depicting the mechanism of Capto Core beads for separating vesicles from free proteins.

FIG. 15 is a schematic representation depicting the experimental procedure for using Capto Core beads in purifying extracellular vesicles.

### DETAILED DESCRIPTION OF THE DISCLOSURE

The present disclosure is based, at least in part, on the discovery of methods for quantifying extracellular vesicles (EVs) in a biological sample by measuring the levels of their transmembrane proteins (tetraspanins), with ultrasensitive protein detection technology, as described herein. Tetraspanins are transmembrane proteins that are highly abundant in many cell types and readily found on EVs (see, e.g., Andreu and Yanez-Mo, Front Immunol. (2014); 5:442, the contents of which are hereby incorporated herein by reference). Tetraspanins are also referred to as “EV markers.”

Although several technologies have been applied to EV detection, single molecule array (Simoa®) technology has a unique set of features that makes it suited for measuring EVs, overcoming several limitations of other methods. In addition to differentiating EVs from other particles (such as lipoproteins or aggregated proteins), Simoa® also offers high dynamic range, throughput and sensitivity. Since techniques such as ELISA employ signal amplification, they can offer high sensitivity in detection of these proteins in EVs. However, the methods of the disclosure provide greatly enhanced sensitivity using Simoa® assays, which convert ELISA assays into a digital readout by distributing single protein-antibody complexes into femtoliter wells and are 100 to 1000 times more sensitive than traditional ELISAs. This is particularly important for the detection of EVs in human biofluids as the levels of EVs and their cargo proteins are often low in clinical samples and difficult to detect with conventional techniques.

As described herein, the present inventors have developed Simoa® assays for the detection of three transmembrane proteins, the tetraspanins CD9, CD63, and CD81. The assays described herein can be used to detect not only these tetraspanins, but other proteins, including transmembrane proteins, present in EVs. These Simoa® assays have been used by the inventors to quantify EVs in human plasma and cerebrospinal fluid (CSF).

In order to use EVs for diagnostic, prognostic, and other purposes, it is necessary to purify EVs for downstream analysis prior to quantification. The Simoa® assays described herein were used to improve size exclusion chromatography (SEC) for EV purification from biological samples, e.g., liquid biological samples such as plasma and CSF, using a resin that has not previously been used for EV purification. Additionally, a high-throughput, columnless method for purification of EVs is also described herein.

Accordingly, the present disclosure is based, at least in part, on the discovery of methods for purifying and quantitating EVs in a biological sample by measuring levels of transmembrane proteins present on the EVs. In some embodiments, the methods of the present disclosure comprise utilizing ultrasensitive Simoa® assays for quantification of EV transmembrane proteins, including, but not limited to, tetraspanins CD9, CD63, and CD81, to measure EVs in a biological sample, e.g., plasma or CSF.

In addition, the present disclosure is based, also in part, on the discovery of methods for purification of EVs from a biological sample using size exclusion chromatography (SEC) with a Sepharose™ resin, e.g., a Sepharose™ CL-6B resin. In some embodiments, Simoa® is used to optimize SEC to optimize and improve SEC in biological samples, including plasma and CSF.

The present disclosure is further based, at least in part, on the discovery of high-throughput methods for purification of EVs from a biological sample using size-exclusion beads, e.g., simple eluate beads, such as, for example, Capto™ Core 700 beads. In some embodiments, the method does not require use of columns for purification of EVs from biological samples. In some embodiments, the purification method is an in-slurry method. In other embodiment, the purification method disclosed herein is a high-throughput method.

The inventors have successfully demonstrated for the very first time, that using the claimed methods of the present application, e.g., purifying EV from a biological sample using size-exclusion beads alone or in combination with one or more additional chromatography steps, e.g., a size exclusion chromatography and/or a cation exchange chromatography, result in a complete removal of the major contaminating protein, albumin, as well as a significant reduction in the level of lipoprotein. This represents a significant improvement over all existing EV purification methods.

The inventors have also demonstrated that the methods of this application confer additional advantages over the commonly used EV purification methods in that a small sample size, e.g., 1 mL, is sufficient for EV purification. In addition, the methods of the present application are extremely efficient in that they can be completed within two hours, and are amenable to high throughput applications. In contrast, the currently existing EV purification methods generally include ultracentrifugation, and density gradient centrifugation steps, which not only are time consuming, but also require special instruments that are not suitable for high throughout methods. Furthermore, the methods of the present application are able to recover at least 2 fold more EV from the plasma and CSF samples when compared to existing methods in the art.

The methods disclosed herein for the purification and quantification of EVs in biological samples can be used in diagnostic and prognostic methods, wherein EV proteins serve as biomarkers for various diseases and disorders. Thus, the present disclosure provides methods and kits for the diagnosis or prognosis of diseases or disorders in a subject by purifying EVs in a biological sample from a subject and detecting and measuring the levels of one or more proteins, e.g., EV biomarkers or tetraspanins, in the EVs purification from the biological sample.

The present disclosure also provides methods of using the EVs purified by methods disclosed herein as therapeutics (e.g., as a drug delivery system) for a wide variety of indications.

### I. Definitions

In order that the present disclosure may be more readily understood, certain terms are first defined. It should also be noted that whenever a value or range of values of a parameter are recited, it is intended that values and ranges intermediate to the recited values are also part of this disclosure.

In the following description, for purposes of explanation, specific numbers, materials, and configurations are set forth in order to provide a thorough understanding of the disclosure. It will be apparent, however, to one having ordinary skill in the art that the disclosure may be practiced without these specific details. In some instances, well-known features may be omitted or simplified so as not to obscure the present disclosure. Furthermore, reference in the specification to phrases such as “one embodiment” or “an embodiment” mean that a particular feature, structure, or characteristic described in connection with the embodiment is included in at least one embodiment of the disclosure. The appearances of phrases such as “in one embodiment” in various places in the specification are not necessarily all referring to the same embodiment.

The articles “a” and “an” are used herein to refer to one or to more than one (i.e., to at least one) of the grammatical object of the article. By way of example, “an element” refers to one element or more than one element.

The term “comprising” or “comprises” is used herein in reference to compositions, methods, and respective component(s) thereof, that are essential to the disclosure, yet open to the inclusion of unspecified elements, whether essential or not.

The term “portion” includes any fraction of a population (e.g., a plurality of capture objects), or region of a protein, such as a fragment of an protein (e.g., about 5%, 10%, 15%, 20%, 30%, 40%, 50%, 60%, 70%, 80%, 90%, 95%, or 99%, fewer amino acids of a full-length antibody.)

As used herein, a “digital enzyme-linked immunosorbent assay” and “single-molecule array assay” refer to an ultrasensitive immunoassay. An example of a digital enzyme-linked immunosorbent assay is the Simoa® digital immunoassay. Simoa® stands for “Single Molecule Array” and is a registered trademark owned by the QUANTERIX corporation of Lexington, Mass., U.S.A. The Simoa® platform provides a 50 to 1000-fold increase in sensitivity for protein detection, compared to ELISA, utilizing a bead-based immunoassay format with multiplex ability that is amenable to implementation in future POC devices. Unlike conventional ELISA methods, Simoa® has the ability to trap single bead-bound molecules in femtoliter-sized wells, allowing for a “digital” readout of each individual bead to determine whether the bead is bound to the target molecule. The digital nature (e.g., “yes-no” or “true-false”) of the result for each well allows an average increase in sensitivity of three orders of magnitude (1,000 times) over conventional ELISA with coefficients of variation (CV) less than about ten percent. Simoa® assays are described in, for example, U.S. Pat. Nos. 8,236,574 and 9,310,360 and related patents, the contents of which are hereby incorporated herein by reference.

As described herein, the Simoa® assays of the present disclosure are capable of highly sensitive detection of transmembrane proteins including tetraspanins which are present in extracellular vesicles (EVs), to thereby quantitate EVs in liquid biological samples such as, but not limited to, plasma and cerebrospinal fluid samples.

The term “antigen (Ag)” or “antigen molecule” as used herein, refers to a molecule that can induce an immune response, such as an antibody response by the host's immune system. An antigen is specifically recognized and bound by antibodies (Ab). In some embodiments, the antigen is a protein, e.g., a transmembrane protein, present in an EV. For example, in some embodiments, the antigen is a tetraspanin. In some embodiments, the tetraspanin is CD9, CD63, or CD81. In some embodiments, an antigen forms an immune complex with the antibody bound to it, which is referred to herein as an “antigen-antibody immunocomplex.”

The term “epitope”, as used herein, refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain.

The terms “immune complex,” “immunocomplex,” and “antigen-antibody immunocomplex,” as used interchangeably herein, refer to a complex, e.g., a non-covalently bound association between an immunoglobulin molecule, such as an antibody molecule, and an antigen, e.g., a tetraspanin.

In the methods described herein, a capture object can bind to an antigen molecule and form a “capture object-antigen molecule complex.” A capture object may also bind to an antigen within an antigen-antibody immunocomplex and form a “complex of the capture object and the antigen-antibody immunocomplex.”

The antigen-antibody immunocomplex can act as a single molecule and may also act as an antigen of its own, for example, a second antibody molecule can bind to the Fc portion of the IgG antibody molecule within the antigen-antibody immunocomplex, and, e.g., detect the antigen-antibody immunocomplex.

The term “capture object,” as used herein, refers to an object comprising a capture probe to which a capture moiety may be conjugated, captured, attached, bound, or affixed. Detection probes or detectable moieties may bind or otherwise associate with a capture object in single molecule array assays as described herein. Suitable capture probes include, but are not limited to, beads (e.g., paramagnetic beads), nanotubes, polymers, particulate suspensions, plates, disks, dipsticks, or the like. In some embodiments, a reaction vessel used in the methods of the disclosure (e.g., a microwell) is capable of holding zero or one capture object.

The term “bead” means a small discrete particle that may be used to capture molecules, e.g., molecules of a certain size. Suitable beads include, but are not limited to, paramagnetic beads, plastic beads, ceramic beads, glass beads, polystyrene beads, methylstryene beads, acrylic polymer beads, carbon graphited beads, titanium dioxide beads, latex or cross-linked dextrans such as Sepharose® beads, cellulose beads, nylon beads, cross-linked micelles, and Teflon® beads. In some embodiments, spherical beads are used, but it is to be understood that non-spherical or irregularly-shaped beads may be used.

In some embodiments, the bead is a “size-exclusion bead.” As used herein, a size-exclusion beads is a bead that excludes molecules that are greater than a certain size from entering the core of the bead and molecules less than a certain size are captured by, or bind to, the core of the bead. In some embodiments, the beads exclude molecules that are the size of extracellular vesicles or greater. In some embodiments, the beads exclude molecules which are greater than the size of a target EV, e.g., greater than about 700 kDa. In some embodiments, the beads comprise an inactive bead exterior or shell. In some embodiments, the shell comprises pores. In some embodiments, the beads contain a ligand-activated core such as an octylamine ligand. In some embodiments, the size-exclusion beads comprise bind-elute resins. In some embodiments, the size-exclusion beads are Capto™ Core resin beads, e.g., Capto™ Core 700 resin beads or Capto™ Core resin 400 beads.

The term “capture moiety,” as used herein, means a moiety that is capable of specifically binding to an antigen molecule. In some embodiments, the antigen is a tetraspanin, e.g., CD9, CD63, or CD81.

A capture moiety may be conjugated, captured, attached, bound, or affixed to a capture probe in a capture object. For example, in some embodiments, a capture moiety is an antibody (e.g., a full-length antibody (e.g., an IgG, IgA, IgD, IgE, or IgM antibody) or an antigen-binding antibody fragment (e.g., an scFv, an Fv, a dAb, a Fab, an Fab′, an Fab′2, an F(ab′)2, an Fd, an Fv, or an Feb)), an aptamer, an antibody mimetic (e.g., an affibody, an affilin, an affimer, an affitin, an alphabody, an anticalin, an avimer, a DARPin, a fynomer, a Kunitz domain peptide, a monobody, or a nanoCLAMP), an antibody IgG binding protein (e.g., protein A, protein G, protein L, and recombinant protein A/G), a polypeptide, a nucleic acid, or a small molecule.

In some embodiments, a capture moiety binds to an antigen present in an EV, e.g., a tetraspanin. In some embodiments, the tetraspanin is CD9, CD63, or CD81. In some embodiments, a capture moiety binds to an antigen-antibody immunocomplex. The capture moiety is coupled to a capture probe, forming a capture object. In some embodiments, a plurality of capture moieties are coupled to a capture probe to form a capture object. In some embodiments, about 1 to about 10,000,000 capture moieties are coupled to a capture probe to form a capture object. For example, in some embodiments, about 1 to about 1,000,000 capture moieties are coupled to a capture probe to form a capture object. In some embodiments, about 500,000 to about 1,000,000 capture moieties are coupled to a capture probe to form a capture object. In some embodiments, about 10,000 to about 500,000 capture moieties are coupled to a capture probe to form a capture object.

The term “detection probe,” as used herein, refers to any molecule, particle, or the like that is capable of specifically binding to a target molecule, e.g., an antigen molecule an antigen present on an EV, e.g., a tetraspanin, or another molecule that binds to or otherwise associates with the target molecule (e.g., an Fc portion of an antibody in an antigen-antibody immunocomplex). For example, in some embodiments, a detection probe is an antibody (e.g., a full-length antibody, such as an IgG, IgA, IgD, IgE, or IgM antibody) or an antigen-binding antibody fragment (e.g., an scFv, an Fv, a dAb, a Fab, an Fab′, an Fab′2, an F(ab′)2, an Fd, an Fv, or an Feb)), an aptamer, an antibody mimetic (e.g., an affibody, an affilin, an affimer, an affitin, an alphabody, an anticalin, an avimer, a DARPin, a fynomer, a Kunitz domain peptide, a monobody, or a nanoCLAMP), a molecularly imprinted polymer, a receptor, a polypeptide, a nucleic acid, or a small molecule.

The term “detectable moiety,” as used herein, refers to a moiety that can produce a detectable signal. For example, in some embodiments, a detectable moiety is, or comprises, an enzymatic label, such as beta-galactosidase, horseradish peroxidase, glucose oxidase, and alkaline phosphatase), a fluorescent label, a radioactive label, or a metal label. In some embodiments, the detectable moiety is beta-galactosidase.

As used herein, a molecule or moiety “specifically binds” to another molecule or moiety with specificity sufficient to differentiate from other components or contaminants of the test sample. In some embodiments, a molecule or moiety specifically binds to another molecule or moiety with an equilibrium dissociation constant (K_{D}) of about 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, 10⁻¹⁴ M, 10⁻¹⁵ M, or lower.

The term “non-covalent affinity binding pair” refers to a pair of moieties that bind and form a non-covalent complex. Exemplary non-covalent affinity binding pairs include, without limitation, biotin-biotin binding protein (e.g., biotin-streptavidin and biotin-avidin), ligand-receptor, antigen-antibody or antigen binding fragment, hapten-anti-hapten, and immunoglobulin (Ig) binding protein-Ig. The members of a non-covalent affinity binding pair may have any suitable binding affinity. For example, the members of an affinity binding pair may bind with an equilibrium dissociation constant (K_{D} or Kd) of about 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, 10⁻¹⁴ M, 10⁻¹⁵ M, or lower.

As used herein, “subject” refers to any animal. In some embodiments, the subject is a human. Other animals that can be subjects include but are not limited to non-human primates (e.g., monkeys, gorillas, and chimpanzees), domesticated animals (e.g., horses, pigs, donkeys, goats, rabbits, sheep, cattle, yaks, alpacas, and llamas), and companion animals (e.g., cats, dogs, hamsters, guinea pigs, rats, mice, and birds.)

As used herein, “biological sample” refers to any biological sample obtained from or derived from a subject. In some embodiments, the biological sample contains EVs. In a preferred embodiment, the biological sample is a liquid biological sample. The term “liquid biological sample,” as used herein, refers to a sample that is substantially in liquid form. In some embodiments, a liquid sample is a body fluid. Body fluids include, e.g., serum (including fresh or frozen), plasma (including fresh or frozen), peripheral blood mononuclear cells, whole blood (including fresh or frozen), cerebrospinal fluid (CSF), synovial fluid, urine, lymph, saliva, semen, sputum, mucous, feces, and vaginal fluid. In some embodiments, the biological sample is a plasma sample. In some embodiments, the biological sample is a CSF sample.

### II. Purification of Extracellular Vesicles

The present disclosure provides methods for the purification of extracellular vesicles (EVs) from circulating proteins and other contaminants present in a sample, e.g., a biological sample such as plasma or cerebrospinal fluid. Methods for purification of EVs using size-exclusion beads and methods for purification of EVs using size exclusion chromatography (SEC) are described herein.

Purification of Extracellular Vesicles with Size-Exclusion Beads

The inventors have shown that size-exclusion beads can “trap” contaminants within the bead, thus enabling purification of extracellular vesicles from a sample, e.g., a liquid biological sample. The disclosed methods are independent of columns and other chromatography equipment and are therefore compatible with high-throughput purification of extracellular vesicles. Thus, the present disclosure provides high-throughput methods for purification of EVs from a biological sample using size-exclusion beads. In some embodiments, the beads exclude molecules which are greater than the size of a target EV, e.g., greater than about 400, 500, 600, or 700 kDa. In some embodiments, the beads comprise an inactive bead exterior or shell. In some embodiments, the shell comprises pores. In some embodiments, the beads contain a ligand-activated core such as an octylamine ligand. In some embodiments, the size-exclusion beads comprise a bind-elute resin. In some embodiments, the size-exclusion beads are Capto™ Core resin beads, e.g., Capto™ Core 700 resin beads or Capto™ Core resin 400 beads.

In some embodiments, the method does not require use of columns for purification of EVs from biological samples.

In some aspects, the disclosure provides methods for purifying extracellular vesicles from a biological sample comprising combining a liquid biological sample containing extracellular vesicles with size-exclusion beads capable of capturing molecules smaller the size of a target EV, e.g., greater than about 700 kDa, to create a mixture, and separating and removing the size-exclusion beads from the mixture, such that the extracellular vesicles remain, or by removing the supernatant from the mixture, thereby purifying the extracellular vesicles.

In some embodiments, the biological sample is obtained from a subject. In preferred embodiments, the biological sample is a liquid biological sample. One skilled in the art will recognize that a biological sample can be, but is not limited to, the following bodily fluids: peripheral blood, plasma, serum, cerebrospinal fluid (CSF), ascites, sputum, saliva, bone marrow, synovial fluid, aqueous humor, amniotic fluid, cerumen, breast milk, broncheoalveolar lavage fluid, semen (including prostatic fluid), Cowper's fluid or pre-ejaculatory fluid, female ejaculate, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates or other lavage fluids. A biological sample may also include the blastocyl cavity, umbilical cord blood, or maternal circulation that may be of fetal or maternal origin. The biological sample may also be a tissue sample or biopsy, from which EVs may be obtained. In a preferred embodiment, the biological sample is a plasma sample. In another preferred embodiment, the liquid biological sample is a CSF sample.

In some embodiments, the methods of the disclosure are performed as in-slurry methods. The size-exclusion beads can be suspended in a buffer to create a slurry. In some embodiments, the size-exclusion beads can be suspended in an equal volume of buffer to create a 50% slurry, or in any volume buffer effective to produce a slurry with an effective amount of size-exclusion beads to purify EVs from a sample. For example, the slurry can be a 20%, 30%, 40%, 50%, 60%, 70%, 80%, or 90% slurry. Any effective buffer can be used to produce the slurry. In some embodiments, PBS buffer is used.

The size-exclusion beads can be mixed with the biological sample in an amount of, for example, 20 μL, 25 μL, 50 μL, 75 μL, 100 μL, 150 μL, 200 μL, 250 μL, 300 μL, 350 μL, or 400 μL per 1 mL of liquid biological sample.

The combination, or mixture, of size-exclusion beads and the sample containing the EVs can be mixed, agitated, or rotated prior to separation and removal of the size-exclusion beads. For example, the mixture can be mixed, agitated, or rotated for about 10, 20, 30, 40, 45, 50, 55, 60, 65, 70, 75 or more minutes prior to separation and removal of the size-exclusion beads. In some embodiments, the mixture is mixed, agitated, or rotated for between about 30 minutes and about 1 hour. In some embodiments, the mixture is mixed, agitated, or rotated for about 45 minutes. The mixture can be mixed, agitated, or rotated at room temperature, or at any temperature conducive to the capture of impurities in the sample by the size-exclusion beads.

The size-exclusion beads can be separated from the mixture using any effective means known in the art. In some embodiments, centrifugation can be used to separate the size-exclusion beads from the mixture. In some embodiments, the mixture is centrifuged at between about 600 g, 700 g, 800 g, 900 g or higher. In some embodiments, the mixture is centrifuged at about 800 g. In some embodiments, the mixture is centrifuged for enough time to adequately separate the size-exclusion beads from remainder of the mixture to obtain the EVs in the supernatant. In some embodiments, the mixture is centrifuged for about 10 minutes, or for about 10, 15, 20, 25, 30, 40 or more minutes.

The size-exclusion beads used in the methods of the disclosure can, in some embodiments, comprise an inactive bead exterior or shell. The exterior of the bead can comprise pores which allow molecules less than a certain size to pass through the exterior shell and be trapped in the core of the bead. In some embodiments, the beads can comprise a core comprising interior of the beads comprise a ligand such as a multimodal ligand such as octylamine ligand. In a preferred embodiment the total shell bead (i.e., shell plus core) thickness is preferably 40-100 microns in diameter, and the shell thickness is preferably 2-10 microns.

In some preferred embodiments, the size-exclusion beads used in the methods of the disclosure comprise an inner porous core and an outer porous shell, wherein the inner core is provided with octylamine ligands and the shell is inactive, and wherein the porosity of the shell and core does not allow entering of molecules larger than about 700 kD.

In some embodiments, the size-exclusion beads comprise a bind-elute resin. In some embodiments, the size-exclusion beads are Capto™ Core bind-elute beads. In some embodiments, the size-exclusion beads are Capto™ Core 700 bind-elute beads. Capto™ Core 700 chromatography resin (GE Healthcare Biosciences AB) comprise octylamine ligands within Capto™ Core 700 ‘beads’, and are designed to have both hydrophobic and positively charged properties that can trap molecules under 700 kilodaltons. Since extracellular vesicles exceed 700 kDa, and since the bead exterior is inactive, Capto Core 700 permits purification of extracellular vesicles by size exclusion. With standard gel filtration (size exclusion chromatography), molecules of smaller size spend more time penetrating pores of the stationary phase, and therefore exhibit higher retention (slower elusion) relative to larger molecules. In contrast, the ligand-activated pores of Capto™ Core 700 have electrostatic and hydrophobic interactions that “capture” molecules under 700 kDa.

In some embodiments, the biological sample, e.g., plasma or CSF, is subjected to a size exclusion chromatography (SEC) column prior to combining with the size-exclusion beads capable of capturing molecules smaller than about 700 kDa. Any size exclusion chromatography resins known in the art are suitable for the methods of the present invention. In some embodiments, the size exclusion chromatography column comprises a stationary phase comprising a 6% cross-linked agarose size exclusion chromatography base matrix. In some embodiments, the 6% cross-linked agarose size exclusion chromatography base matrix is a Sepharose™ CL-6B resin.

In some embodiments, the biological sample, e.g., plasma or CSF, is further subjected to a cation exchange chromatography resin after the size exclusion chromatography column and prior to combining with the size-exclusion beads capable of capturing molecules smaller than about 700 kDa. Any cation exchange chromatography resins known in the art are suitable for the methods of the present invention. Exemplary cation exchange chromatography resin include, but are not limited to, sulpfhydryl, sulfonate, sulfate, carboxymethyl, sulfoethyl, sulfopropyl, phosphate and sulfonate. In some embodiment, the cation exchange chromatography resin is Fractogel® EMD-SO₃⁻ resin.

The methods for EV purification disclosed herein can also be optimized for high-throughput applications.

Purification of Extracellular Vesicles with Size Exclusion Chromatography

The present disclosure also provides methods for purification of EVs from a biological sample using size exclusion chromatography (SEC) columns with a stationary phase material comprising an agarose size exclusion chromatography base matrix, for example, a base matrix comprising 6% agarose content. In some embodiments, the stationary phase material is a 6% cross-linked agarose size exclusion chromatography base matrix. In some embodiments, the stationary phase material is a Sepharose™ resin, e.g., a Sepharose™ cross-linked resin such as Sepharose™ CL-6B resin. In SEC, a porous stationary phase is utilized to sort macromolecules and particulate matters according to their size. Components in a sample with small hydrodynamic radii are able to pass through the pores, thus resulting in late elution. Components with large hydrodynamic radii, including EVs, are excluded from entering the pores. The SEC columns used in the methods of the invention yield significant improvements in EV yield as compared to other columns at a fraction of the cost. In particular, it has been found that Sepharose™ CL-6B yields considerably higher levels of EVs than Sepharose™ CL-2B or Sepharose™ CL-4B. This is due to the fact that Sepharose™ CL-6B beads have a smaller average pore size, leading to a lower probability that EVs will enter the beads.

Accordingly, the present disclosure provides methods for purifying extracellular vesicles from a biological sample, the method comprising providing a SEC column comprising a stationary phase material comprising a 6% cross-linked agarose size exclusion chromatography base matrix, e.g., a Sepharose™ CL-6B resin, introducing a sample comprising extracellular vesicles into the column, flowing the sample through the stationary phase material, and collecting fractions containing extracellular vesicles from the SEC column, thereby purifying the extracellular vesicles.

In some embodiments, the sample is a biological sample is obtained from a subject. In preferred embodiments, the biological sample is a liquid biological sample. One skilled in the art will recognize that a biological sample can be, but is not limited to, the following bodily fluids: peripheral blood, plasma, serum, cerebrospinal fluid (CSF), ascites, sputum, saliva, bone marrow, synovial fluid, aqueous humor, amniotic fluid, cerumen, breast milk, broncheoalveolar lavage fluid, semen (including prostatic fluid), Cowper's fluid or pre-ejaculatory fluid, female ejaculate, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates or other lavage fluids. A biological sample may also include the blastocyl cavity, umbilical cord blood, or maternal circulation that may be of fetal or maternal origin. The biological sample may also be a tissue sample or biopsy, from which EVs may be obtained. In a preferred embodiment, the biological sample is a plasma sample. In another preferred embodiment, the liquid biological sample is a CSF sample.

In other embodiments, the SEC column is a 5 mL, 7 mL, 10 mL, 12 mL, 15 mL, 20 mL, or 25 mL volume column. In other embodiments, the SEC column is a 5 mL to 25 mL volume column, although columns outside of these ranges can also be used. In some embodiments, the SEC column is a 10 mL volume column. In other embodiments, the SEC column is a 20 mL volume column.

The SEC column comprising a 6% cross-linked agarose size exclusion chromatography base matrix, e.g., Sepharose™ CL-6B resin can be prepared by first washing the resin prior to addition to the column. In some embodiments, the 6% cross-linked agarose size exclusion chromatography base matrix, e.g., a Sepharose™ CL-6B resin is washed in buffer, e.g., PBS, prior to preparation of the column. The resin can be washed multiple times prior to preparation of the column. In some embodiments, the 6% cross-linked agarose size exclusion chromatography base matrix, e.g., Sepharose™ CL-6B resin is washed four or more times in buffer prior to preparation of the column.

Once the resin is washed, it can be added to a suitable column, e.g., a 10 mL or 20 mL column. In one embodiment, the SEC column comprises a housing having at least one wall defining a chamber having an entrance and an exit. In some embodiments, the stationary phase is washed prior to the column comprising the 6% cross-linked agarose size exclusion chromatography base matrix, e.g., Sepharose™ CL-6B resin is washed prior to introducing the sample comprising extracellular vesicles into the column. In some embodiments, the stationary phase is washed with PBS.

Fractions containing EVs can be collected from the SEC column, thereby purifying EVs from the liquid biological sample. For example, fractions 6-21 or fractions 12-27 can be collected, depending on the size of the column. In some embodiments, fractions 6-21 can be collected 10 mL columns. In other embodiments, fractions 12-27 can be collected for 20 mL columns. A smaller number of fractions can also be collected from the SEC column. For example, higher purity of EVs could also be achieved by taking a smaller number of the fractions (e.g. 7-9 instead of 7-10), albeit with lower yield.

While not necessary, following purification of EVs using any of the methods of the present disclosure, the purified extracellular vesicles can be further purified by any means known in the art. In addition, the methods for purification of EVs as described herein can be combined with each other, and with other EV purification methods known in the art. For example, in some embodiments, cation exchange chromatography, size exclusion chromatography, such as gel permeation columns, centrifugation or density gradient centrifugation, and filtration methods can be used in combination with the methods of the disclosure. As another example, the EV purification methods of the disclosure can be used differential centrifugation, anion exchange and/or gel permeation chromatography, sucrose density gradients, organelle electrophoresis, magnetic activated cell sorting (MACS), or with a nanomembrane ultrafiltration concentrator.

In some embodiments, the fractions collected from the SEC column are further subjected to a cation exchange chromatography resin. The cation exchange chromatography resin comprises a stationary phase comprising a functional group selected from the group consisting of sulpfhydryl, sulfonate, sulfate, carboxymethyl, sulfoethyl, sulfopropyl, phosphate and sulfonate. In some embodiments, the cation exchange chromatography resin is Fractogel® EMD-SO₃⁻ resin.

In some embodiments, the fractions collected from the SEC column are further subjected to a size-exclusion beads capable of capturing molecules smaller than about 700 kDa. In some embodiments, the size-exclusion beads are Capto™ Core 700. In another embodiment, the fractions collected from the SEC column are further subjected to a cation exchange chromatography resin and a size-exclusion beads capable of capturing molecules smaller than about 700 kDa.

In some embodiments, the methods of the present application recover at least about 1.5 fold, about 2 fold, about 2.5 fold, about 3 fold, about 3.5 fold, about 4 fold, about 4.5 fold, about 5 fold, about 6 fold, about 7 fold, about 8 fold, about 9 fold, about 10 fold, or more EV from the samples, e.g., plasma and CSF samples, when compared to existing methods in the art.

Highly abundant proteins, such as albumin and immunoglobulins, may hinder purification of exosomes from a biological sample. Therefore, the methods of the disclosure may be used with a system that utilizes multiple antibodies that are specific to the most abundant proteins found in blood. Such a system can remove up to several proteins at once, thus unveiling the lower abundance species such as cell-of-origin specific exosomes. Other known methods for EV purification include high abundant protein removal methods as described in Chromy et al. J. Proteome Res 2004; 3: 1120-1127. In another embodiment, the purification of EVs from a biological sample may also be enhanced by removing serum proteins using glycopeptide capture as described in Zhang et al, Mol Cell Proteomics 2005; 4: 144-155.

### III. Quantifying Extracellular Vesicles

The present disclosure provides methods for quantifying extracellular vesicles (EVs) in a sample, e.g., a liquid biological sample such as a plasma or cerebrospinal fluid sample. In some embodiments, the EVs are quantified by detecting transmembrane proteins on the EVs. For example, the methods of the disclosure describe detection of tetraspanins, which are transmembrane proteins that are highly abundant in many cell types and readily found on EVs.

In some embodiments, the methods of the disclosure comprising detection and/or quantification of one or more EV transmembrane proteins, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34 or more transmembrane proteins, including one or more of the tetraspanins listed in Table 1, below. In some embodiments, the tetraspanins detected are CD9, CD63, and/or CD81.

TABLE 1

| Protein | Aliases |
|---|---|
| TSPAN1 | TSP-1 |
| TSPAN2 | TSP-2 |
| TSPAN3 | TSP-3 |
| TSPAN4 | TSP-4, NAG-2 |
| TSPAN5 | TSP-5 |
| TSPAN6 | TSP-6 |
| TSPAN7 | CD231/TALLA-1/A15 |
| TSPAN8 | CO-029 |
| TSPAN9 | NET-5 |
| TSPAN10 | OCULOSPANIN |
| TSPAN11 | CD151-like |
| TSPAN12 | NET-2 |
| TSPAN13 | NET-6 |
| TSPAN14 | |
| TSPAN15 | NET-7 |
| TSPAN16 | TM4-B |
| TSPAN17 | |
| TSPAN18 | |
| TSPAN19 | |
| TSPAN20 | UP1b, UPK1B |
| TSPAN21 | UP1a, UPK1A |
| TSPAN22 | RDS, PRPH2 |
| TSPAN23 | ROM1 |
| TSPAN24 | CD151 |
| TSPAN25 | CD53 |
| TSPAN26 | CD37 |
| TSPAN27 | CD82 |
| TSPAN28 | CD81 |
| TSPAN29 | CD9 |
| TSPAN30 | CD63 |
| TSPAN31 | SAS |
| TSPAN32 | TSSC6 |
| TSPAN33 | |

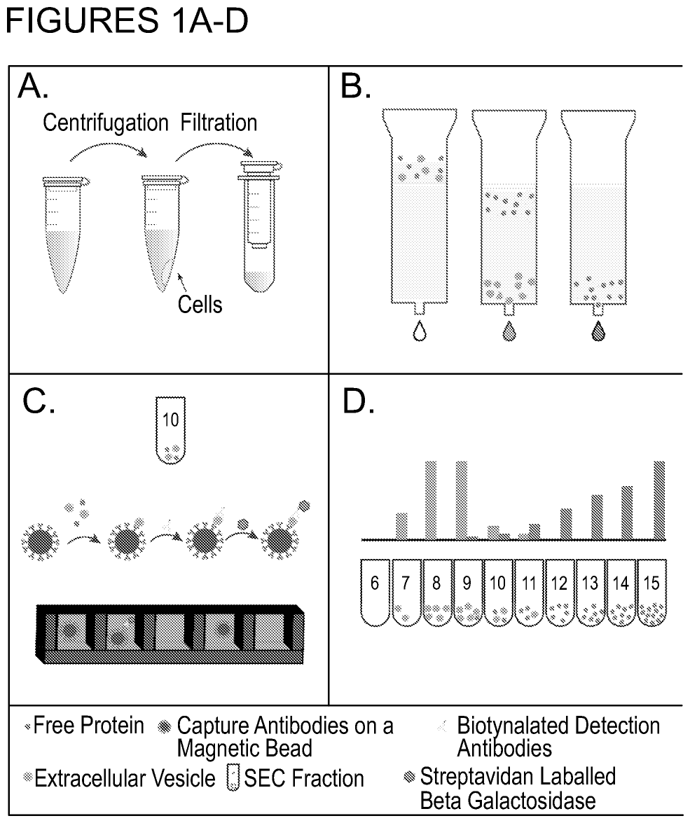

The methods disclosed herein, e.g., the Simoa® methods, can be used to quantify EVs after or in combination with any EV purification method, including one or more of the purification methods disclosed herein. In addition, the experimental framework disclosed herein can be applied to evaluate new EV purification methods in plasma or CSF, or applied to any other biological fluid, such as, but not limited to, urine or saliva. Simoa® has been used to quantify free proteins, but the inventors have developed methods to apply Simoa® to EVs by immuno-isolating single vesicles into microwell arrays with antibodies to transmembrane proteins on their surface (see FIGS. 1A-D). As in conventional ELISA, Simoa® uses two antibodies: a capture antibody conjugated to a magnetic bead, and a detector antibody conjugated to an enzyme. But unlike in traditional ELISA, individual immuno-complexes are isolated into femtoliter wells that fit only one bead per well. In a given reaction, there are many more antibody-bound beads than target proteins, and therefore Poisson statistics dictate that only a single immuno-complex is present per well. This allows for counting “on wells” as individual proteins, or, in the case of EVs, antigen-positive vesicles. Thus, Simoa® can count single antigen-positive EVs with a specific transmembrane protein or combination of transmembrane proteins. The ratio of “on wells” to “off wells” can then be mapped against a standard calibration curve of recombinant protein of known concentration.

The quantification and purification methods disclosed herein can also be applied to the study of EVs in cell culture. The sensitivity and high-throughput nature of Simoa® allows for relative EV quantification in cell culture media from small numbers of cells. Thus, for example, EVs can be quantified after gene editing to study EV biogenesis, or to determine the effect of various cellular perturbations on EV levels. Additionally, the quantification and purification methods disclosed herein can be used to study EV heterogeneity of single EVs.

As sensitivity of EV detection and specificity in differentiating EVs from protein aggregates and lipoproteins are obstacles in all EV studies, ultrasensitive protein detection of EV surface proteins is applicable to both the study of basic EV biology and the development of diagnostic methods. Thus, the disclosure also provides methods for diagnosing or prognosing a disease or disorder in a subject based on the detected EV proteins, and treating the subject diagnosed with the disease or disorder.

The methods of the disclosure include detecting a single antigen molecule, or a single antigen-antibody immunocomplex, in a subject sample. The methods include contacting a subject sample containing the antigen molecule or the single antigen-antibody immunocomplex with a plurality of capture objects, wherein each capture object comprises a capture probe coupled to a plurality of capture moieties that specifically bind to an antigen, wherein the subject sample and the plurality of capture objects are contacted at a ratio that allows for a single antigen molecule to bind to a single capture object; incubating the capture objects and the antigen molecule or antigen-antibody immunocomplex for a sufficient time to allow binding of the plurality of capture objects to the antigen molecule or the single antigen-antibody immunocomplex contained in the sample, thereby creating a capture object-antigen molecule complex or a complex of capture object and antigen-antibody immunocomplex; contacting the complex from the previous step with a plurality of detection probes, wherein the detection probes bind to the antigen molecule within the capture object-antigen molecule complex or the Fc portion of the antibody of the antigen-antibody immunocomplex; labeling the product with a detectable moiety; and detecting the detectable moiety.

The methods described herein can be used to detect any protein, e.g., transmembrane protein, in an EV. Exemplary proteins include, but are not limited to, CD9, CD63, and/or CD81. Such tetraspanins are described in, for example, Maria Yanex-Mo, et al., J. of Extracellular Vesicles, vol. 4, 2015; Richard J Simpson, et al. Expert Review of Proteomics (2009) 6:3, 267-283; and Helmler, J. Cell Biol. (2001) 155(7): 1103-1108, the entire contents of each of which are incorporated herein by reference.

The capture moieties on the capture objects specifically recognize and bind to an epitope on an antigen, e.g., a tetraspanin. In some embodiments, the methods of the disclosure can be used to detect a single antigen molecule. In some embodiments, the methods of the disclosure can be used to detect multiple antigen molecules. In some embodiments, different epitopes on the same antigen molecule can be detected simultaneously by different groups of capture objects.

Furthermore, the methods described herein can be used to detect any antigen-antibody immunocomplex formed by an antigen. Exemplary antigens that can be detected in the form of antigen-antibody immunocomplex include, but are not limited to, tetraspanins such as, but not limited to, CD9, CD63, and CD81 and those listed in Table 1.

The capture moieties on the capture objects specifically recognize and bind to an epitope on an antigen that is part of an antigen-antibody immunocomplex. In some embodiments, the methods of the disclosure can be used to detect a single antigen-antibody immunocomplex. In some embodiments, the methods of the disclosure can be used to detect multiple antigen-antibody immunocomplexes. In some embodiments, different epitopes on the same antigen-antibody immunocomplex can be detected simultaneously by different groups of capture objects.

The antigen molecules or antigen-antibody immunocomplexes are contacted and captured by a plurality of capture objects. Each capture object comprises a capture probe coupled to a plurality of capture moieties that specifically bind to an antigen molecule or an antigen-antibody immunocomplex.

The capture probe in the capture object supports and is coupled to a plurality of capture moieties (e.g., anti-tetraspanin antibodies) that specifically bind to the tetraspanin antigen or tetraspanin antigen-antibody immunocomplex. Any suitable capture probe can be used in the context of the disclosure, including, without limitation, beads (e.g., paramagnetic beads), nanotubes, polymers, plates, disks, dipsticks, or the like. Suitable beads include, but are not limited to, paramagnetic beads, plastic beads, ceramic beads, glass beads, polystyrene beads, methylstyrene beads, acrylic polymer beads, carbon graphited beads, titanium dioxide beads, latex or cross-linked dextrans such as SEPHAROSE™ beads, cellulose beads, nylon beads, cross-linked micelles, and TEFLON® beads.

In some embodiments, the capture moiety is covalently or non-covalently coupled to the capture probe (e.g., paramagnetic bead) to form the capture object. The capture moieties may be covalently linked to the bead using any suitable conjugation approach known in the art or described herein. The immobilized capture moieties may be non-covalently linked to the capture probes, for example, by forming a non-covalent affinity binding pair.

The plurality of capture moieties specifically bind to the tetraspanin antigen molecule or tetraspanin antigen-antibody immunocomplex. In some embodiments, the capture moiety is an antibody. In some embodiments, the capture moiety is an anti-tetraspanin antibody. The anti-tetraspanin antibody is obtained from a human, a recombinant bacterium, a non-human, non-primate mammal such as a rabbit, a mouse, a rat, a sheep, a goat, or other animal. In some anti-tetraspanin antibody is a rabbit IgG molecule specific to an antigen. In some embodiments, the capture moiety is a full-length antibody, an antigen-binding fragment of an antibody, or an antibody mimetic. In some embodiments, the full-length antibody is an IgG, IgA, IgD, IgE, or IgM antibody. In some embodiments, the antigen-binding fragment is an scFv, an Fv, a dAb, a Fab, an Fab′, an Fab′2, an F(ab′)2, an Fd, an Fv, or an Feb. In some embodiments, the antibody mimetic is an affibody, an affilin, an affimer, an affitin, an alphabody, an anticalin, an avimer, a DARPin, a fynomer, a Kunitz domain peptide, a monobody, or a nanoCLAMP.

The capture object may be detectably labeled to allow for detection of different epitopes on the same antigen. For example, in multiplexed assays, a first population of capture objects may be detectably labeled with a first label, and a second population of capture objects may be detectably labeled with a second label, such that the first population and the second population are distinguishable (also referred to herein as “distinguishably labeled”). In some embodiments, the capture probe in the capture object is detectably labeled. In some embodiments, the capture moiety in the capture object is detectably labeled.

Any suitable label can be used. For example, the label may be a reporter dye (e.g., a fluorescent dye, a chromophore, or a phospho), or a mixture thereof). By varying both the composition of the mixture (i.e., the ratio of one dye to another) and the concentration of the dye (leading to differences in signal intensity), matrices of unique tags may be generated. Capture probes (e.g., beads) can be labeled using any suitable approach, for example, by covalently attaching the label (e.g., a dye) to the surface of the capture probes, or alternatively, by entrapping the label (e.g., a dye) within the capture object. Such dyes may be, for example, covalently attached to the surface of a capture probe (e.g., a bead), for example, using any of the conjugation approaches described above or herein. Suitable dyes for use in the disclosure include, but are not limited to, ALEXA FLUOR® dyes, CY® dyes, DYLIGHT® dyes, fluorescein, rhodamine, tetramethylrhodamine, eosin, erythrosin, coumarin, methyl-coumarins, pyrene, Malachite green, fluorescent lanthanide complexes, including those of europium 5 and terbium, stilbene, Lucifer Yellow, CASCADE BLUE™ TEXAS RED®, and others known in the art (e.g., as described in The Molecular Probes Handbook, 11th Ed., 2010).

The total number of capture moieties (e.g., antibody molecules) coupled to a capture probe (e.g., bead) on a single capture object may be between about 1 and about 10,000,000, between about 50,000 and about 5,000,000, or between about 100,000 and about 1,000,000. In some embodiments, the total number of capture objects provided is at least about 10,000, at least about 50,000, at least about 100,000, at least about 1,000,000, at least about 5,000,000, at least about 10,000,000, at least about 100,000,000, at least about 200,000,000, at least about 300,000,000, at least about 400,000,000, at least about 500,000,000, at least about 600,000,000, at least about 700,000,000, at least about 800,000,000, at least about 900,000,000, at least about 1,000,000,000, at least about 2,000,000,000, at least about 3,000,000,000, at least about 4,000,000,000, or at least about 5,000,000,000.

The total number of capture objects provided for contacting the subject sample containing a target molecule may be between about 1,000 to about 5,000,000 capture objects, e.g., about 1000, about 10,000, about 20,000, about 30,000, about 40,000, about 50,000, about 60,000, about 70,000, about 80,000, about 90,000, about 100,000, about 200,000, about 300,000, about 400,000, about 500,000, about 600,000, about 700,000, about 800,000, about 900,000, about 1,000,000, about 2,000,000, about 3,000,000, about 4,000,000, or about 5,000,000 capture objects. In some embodiments, any of the methods disclosed herein may involve contacting the sample with about 10,000 to about 5,000,000 capture objects, about 10,000 to about 4,000,000 capture objects, about 10,000 to about 3,000,000 capture objects, about 10,000 to about 2,000,000 capture objects, about 10,000 to about 1,000,000 capture objects, about 10,000 to about 500,000 capture objects, about 10,000 to about 400,000 capture objects, about 10,000 to about 300,000 capture objects, about 10,000 to about 200,000 capture objects, or about 10,000 to about 100,000 capture objects.

Contacting the subject sample containing the target molecule (tetraspanin antigen molecule or tetraspanin antigen-antibody immunocomplex) with the capture object is performed for a duration of time and under conditions favorable to binding of the tetraspanin antigen molecule or tetraspanin antigen-antibody immunocomplex with the capture moiety, such as the capture antibody, to the antigen.

The capture objects and the subject sample containing the target molecule (e.g., the antigen or the antigen-antibody immunocomplex) are incubated to allow binding of the capture objects to the target molecule. In some embodiments, the incubation can be performed for less than about 1 minute; about 1 minute to about 8 hours, e.g., about 1 minute about 5 minutes, about 10 minutes, about 20 minutes, about 30 minutes, about 40 minutes, about 50 minutes, about 60 minutes, about 2 hours, about 3 hours, about 4 hours, about 5 hours, about 6 hours, about 7 hours, or about 8 hours. In some embodiments, the incubation of the capture object with the subject sample containing the tetraspanin antigen or antigen-antibody immunocomplex is about 15 minutes to about 35 minutes.

The capture objects and the subject sample containing the tetraspanin antigen or tetraspanin antigen-antibody immunocomplex may be incubated under a physiological condition that maintains the secondary and tertiary protein structure and the conformations of the epitopes on antigen molecules or antigen-antibody immunocomplexes. In some embodiments, the incubation is performed under a pH of about 5, about 6, about 7, or about 8. In some embodiments, the incubation is performed at a temperature of about 4° C. to about 37° C. In some embodiments, the incubation is performed at about 4° C. In some embodiments, the incubation is performed at about 25° C. In some embodiments, the incubation is performed at about 37° C.

To allow for single molecule detection, the capture objects are incubated with the target molecule (e.g., the tetraspanin antigen molecule or tetraspanin antigen-antibody immunocomplex) at a ratio that allows for a single target molecule to bind to a single capture object. In particular, the amount of the capture objects may exceed the amount of the tetraspanin antigen molecules or the tetraspanin antigen-antibody immunocomplexes contained in the subject sample to allow for a distribution of the target molecule on the capture object that at least some of the capture objects bind to tetraspanin antigen molecules or tetraspanin antigen-antibody immunocomplexes and a portion of the capture objects do not bind with any antigen molecule or any antigen-antibody immunocomplex. For example, the ratio of capture object and target molecule can be about 1:1 to about 50:1. In some embodiments, the ratio of capture object and target molecule is about 1:1, about 2:1, about 3:1, about 4:1, about 5:1, about 10:1, about 15:1, about 20:1, about 25:1, about 30:1, about 35:1, about 40:1, about 45:1, about 50:1. In some embodiments, the ratio between capture objects and the antigen molecules or antigen-antibody immunocomplexes in the incubation is about 50:1.

In some embodiments, at least some of the capture objects bind to antigen molecules or antigen-antibody immunocomplexes and a portion of the capture objects do not bind with any antigen molecule or any antigen-antibody immunocomplex. In some embodiments, at least 10%, 20%, 30%, 40%, 50%, 60%, 70%, 80%, or 90% of the capture objects are not bound to any antigen molecule or any antigen-antibody immunocomplex. In preferred embodiments, at least 80% of the capture objects are not bound to any antigen molecule or antigen-antibody immunocomplex.

Upon contacting and incubation, the capture objects and the tetraspanin antigen molecule form capture object-antigen molecule complexes. In some embodiments, at least 90%, 91%, 92%, 93%, 94%, 95%, 96%, 97%, 98%, 99% of the object-antigen molecule complexes have a single tetraspanin antigen molecule bound to a single capture moiety on each capture object.

Upon contacting and incubation, the capture objects and the tetraspanin antigen-antibody immunocomplex form complexes of capture object and antigen-antibody immunocomplexes. In some embodiments, at least 90%, 91%, 92%, 93%, 94%, 95%, 96%, 97%, 98%, 99% of the complexes of capture object and antigen-antibody immunocomplexes have a single tetraspanin antigen-antibody immunocomplex bound to a single capture moiety on each capture object.

The methods disclosed herein provides an advantageously high dynamic range of detecting and quantifying antigen molecules and antigen-antibody immunocomplexes in a subject sample. In some embodiments, the ratio of capture object and target molecule can be adjusted to allow more than one antigen molecules or antigen-antibody immunocomplexes to bind to a capture object, and the antigen molecules or antigen-antibody immunocomplexes are detected and quantified according to the intensity of signals generated in the later steps described herein.

In some embodiments, the plurality of capture objects and the antigen-antibody immunocomplexes are incubated at a ratio of about 1:100 to about 50:1. In one embodiment, the plurality of capture objects and the antigen-antibody immunocomplexes are incubated at a ratio of about 1:90, about 1:80, about 1:70, about 1:60, about 1:50, about 1:40, about 1:30, about 1:20, about 1:10, or about 1:5. In one embodiment, the plurality of capture objects and the antigen-antibody immunocomplexes are incubated at a ratio of about 1:30.

In one embodiment, at least 70% of the capture objects are bound to one or more antigen-antibody immunocomplexes.

In some embodiments, more than one group of capture objects are incubated with a subject sample containing the target molecule (e.g., the tetraspanin antigen molecule or tetraspanin antigen-antibody immunocomplex). For example, different capture objects can be designed so that they specifically bind to different epitopes on each of the same antigen molecules or each of the same antigen-antibody immunocomplexes. By detecting different epitopes simultaneously on the same antigen, the methods of the disclosure have significant advantages in accuracy and sensitivity, compared to traditional assays.

In some embodiments, the capture objects that specifically bind to a first epitope on an antigen (or antigen-antibody immunocomplex) are distinguishably labeled from capture objects that specifically bind to a second epitope on an antigen (or antigen-antibody immunocomplex) so that different labels can be distinguished. In some embodiments, more than one group of distinguishably labeled capture objects are incubated with the subject sample containing the target molecule (e.g., the tetraspanin antigen molecule or tetraspanin antigen-antibody immunocomplex), and each group of capture objects specifically bind to one distinct epitope on an antigen molecule or antigen-antibody immunocomplex expressed by tetraspanin. In some embodiments, each group of distinguishably labeled capture objects are distinguishably detected, therefore distinct epitopes on the same antigen are distinguishably detected. In some embodiments, three groups of capture objects are incubated with the subject sample containing the target molecule (e.g., the tetraspanin antigen molecule or tetraspanin antigen-antibody immunocomplex).

The methods may further comprise a washing step after the incubation of capture objects with the subject sample containing the target molecule (e.g., the tetraspanin molecule or tetraspanin antigen-antibody immunocomplex). The washing step removes excess subject sample and unbound tetraspanin antigen molecules or tetraspanin antigen-antibody immunocomplexes. In some embodiments, the capture objects are washed with a buffered solution. In some embodiments, the buffered solution is phosphate-buffered saline (PBS).

Following the washing step, the capture object-antigen molecule complex, or the complex of capture object with antigen-antibody immunocomplex is contacted with a plurality of detection probes (e.g., detection antibodies).

A detection probe, such as a detection antibody, specifically binds to an tetraspanin antigen molecule or an tetraspanin antigen-antibody immunocomplex, and is linked to a first member of an affinity binding pair, wherein the detectable moiety is linked to the second member of the affinity binding pair.

Any suitable detection probe may be used in the context of the present disclosure. For example, in some embodiments, the detection probe is an antibody (e.g., a full-length antibody (e.g., an IgG, IgA, IgD, IgE, or IgM antibody) or an antigen-binding antibody fragment (e.g., an scFv, an Fv, a dAb, a Fab, a Fab′, an Fab′2, an F(ab′)2, an Fd, an Fv, or an Feb)), an aptamer, an antibody mimetic (e.g., an affibody, an affilin, an affimer, an affitin, an alphabody, an anticalin, an avimer, a DARPin, a fynomer, a Kunitz domain peptide, a monobody, or a nanoCLAMP), a molecularly-imprinted polymer, a receptor, a polypeptide, a nucleic acid, or a small molecule. In some embodiments, the detection probe is covalently or non-covalently linked to a detectable moiety. In some embodiments, the non-covalent affinity binding pair is biotin-streptavidin, biotin-avidin, ligand-receptor, antigen-antibody, or antibody binding protein antibody. In some embodiments, the non-covalent affinity binding pair is biotin-streptavidin. In some embodiments, the detection probe is biotinylated. In some embodiments, the detection probe is biotinylated and the detectable moiety is conjugated with a streptavidin.

A detection antibody is contacted with the capture object-target molecule complex (wherein the capture object is bound to the tetraspanin antigen or the tetraspanin antigen-antibody immunocomplex). In some embodiments, for direct detection of non-complexed tetraspanin antigens in the sample, the detection antibody comprises a biotinylated anti-tetraspanin antibody. In some embodiments, for detection of a tetraspanin antigen-antibody immunocomplex, the detection antibody comprises a biotinylated anti-IgG antibody, such as mouse anti-human IgG antibody, rat anti-human IgG antibody, goat anti-human IgG antibody, sheep anti-human IgG antibody, horse anti-human IgG antibody, and rabbit anti-human IgG antibody.

The methods of the disclosure may further comprise labeling the detection probe-bound, capture object-target molecule complex (wherein the capture object is bound to the tetraspanin antigen or the tetraspanin antigen-antibody immunocomplex) with a detectable moiety. The detectable moiety is linked to a second member of an affinity binding pair and can be linked to the detection probe-bound, capture object-target molecule complex via the affinity binding pair.

In some embodiments, the detectable moiety is, or comprises an enzymatic label (e.g., beta-galactosidase, horseradish peroxidase, glucose oxidase, and alkaline phosphatase), a fluorescent label, a radioactive label, or a metal label. For example, in some embodiments, an enzymatic label generates a species (for example, a fluorescent product) that is either directly or indirectly detectable optically. In some embodiments, the method includes detecting a product of an enzymatic reaction as an indication of the presence of the enzymatic label. In some embodiments, the product of the enzymatic reaction is detected upon its release from the enzymatic label in a zone around the discrete site where the enzyme and/or antigen (or antigen-antibody complex, depending on the embodiment) is located (e.g., in a microwell on an array as described herein, e.g., a Simoa® array). In some embodiments, the detectable label on a detectable moiety is β-galactosidase, the enzyme substrate added to the array can be a β-galactosidase substrate such as resorufin-β-D-galactopyranoside or fluorescein di(β-dgalactopyranoside). Additional examples include a variety of enzymatic labels or colored labels (for example, metallic nanoparticles (e.g., gold nanoparticles), semiconductor nanoparticles, semiconductor nanocrystals (e.g., quantum dots), spectroscopic labels (for example, fluorescent labels), and radioactive labels) may be used in the methods described herein. The presence of the detectable moiety can be detected using suitable detection systems, for example, optical detectors (for example, intensified CCD cameras), or any other suitable detectors known in the art.

The methods of the disclosure further include determining the concentration of the tetraspanin antigen molecule or tetraspanin antigen-antibody immunocomplex, for example, by determining the number of detectable moieties bound to the capture object complexes from the previous steps.

The number of the detectable moieties may be determined using the ultrasensitive Single Molecule Array (Simoa®) technology. Simoa® is a registered trademark owned by the Quanterix Corporation (Lexington, Mass., U.S.A.). As defined herein, Simoa® platform offers a 50 to 1000-fold increase in sensitivity for protein detection, compared to ELISA, utilizing a bead-based immunoassay format with multiplex ability that is amenable to implementation in future POC devices. Unlike conventional ELISA methods, Simoa® traps single bead-bound molecules in femtoliter-sized wells and provides a “digital” readout of each individual bead to determine whether the bead is bound to the target molecule. The digital nature (e.g., “yes-no” or “true-false”) of the result for each well creates an average increase in sensitivity of three orders of magnitude—1,000 times—over conventional ELISA with coefficients of variation (CV) less than about ten percent.

In some embodiments, a detecting step includes single-molecule detection of the detectable moieties. For example, in some embodiments, a detection step occurs in an array of microwells, wherein the microwells are capable of holding zero or one capture object. For example, the methods may involve placing the mixture from the previous steps in a plurality of reaction vessels (e.g., microwells). The array may be as described herein or as described, for example, in WO 2014/183096; WO 2010/039179, WO 2009/029073; US 2018/0136203; US 2018/0017552; US 2018/0003703; US 2015/0355182; US 2015/0353997; US 2010/0075355; US 2010/0075439; U.S. Pat. No. 8,846,415; or U.S. Pat. No. 9,482,662, which are incorporated herein by reference in their entirety. In some embodiments, the array is a Quanterix® single molecule array (e.g., Simoa®®.)

In some embodiments, the methods described herein may utilize a plurality or an array of reaction vessels (e.g., microwells) to determine the presence or concentration of one or more target molecules (e.g., tetraspanin antigen molecules or tetraspanin antigen-antibody immunocomplexes). An array of reaction vessels allows a fluid sample to be partitioned into a plurality of discrete reaction volumes during one or more steps of a method. In some embodiments, the reaction vessels may all have approximately the same volume. In other embodiments, the reaction vessels may have differing volumes.

The reaction vessels (e.g., microwells) may have any suitable volume. The microwells may have a volume of about 10 attoliter (aL) to about 10 picoliters (pL). In some embodiments, the microwells have a volume of about 40 femtoliters. In some embodiments, the microwells have a volume of about 10 femtoliters-1 picoliter. In some embodiments, the microwells have a volume of about 10 femtoliters-500 femtoliters. In some embodiments, the microwells have a volume of about 30-100 femtoliters.

For embodiments employing an array of reaction vessels (e.g., microwells), any suitable number of reaction vessels (e.g., microwells) can be used. Arrays containing from about 2 to many billions of reaction vessels can be made by utilizing a variety of techniques and materials. Increasing the number of reaction vessels in the array can be used to increase the dynamic range of an assay or to allow multiple samples or multiple types of molecules to be assayed in parallel. For example, to allow simultaneous detection and quantification of multiple epitopes of the same antigen molecule or antigen-antibody immunocomplex. In some embodiments, the array comprises between one thousand and one million reaction vessels per sample to be analyzed.

In some cases, the array comprises greater than one million reaction vessels. In some embodiments, the array comprises between about 1,000 and about 50,000, between about 1,000 and about 1,000,000, between about 1,000 and about 10,000, between about 10,000 and about 100,000, between about 100,000 and about 1,000,000, between about 1,000 and about 100,000, between about 50,000 and about 100,000, between about 20,000 and about 80,000, between about 30,000 and about 70,000, between about 40,000 and about 60,000, or about 50,000, reaction vessels.

Individual reaction vessels may contain a binding surface. The binding surface may comprise essentially the entirety or only a portion of the interior surface of the reaction vessel or may be on the surface of another material or object that is confined within the reaction vessel, such as, for example, the complex of the capture object and the antigen molecule or the antigen-antibody immunocomplex. For example, in some embodiments, a reaction vessel may be any reaction vessel described in WO 2009/029073, which is incorporated herein by reference in its entirety.

The methods may further include sealing the microwells. At least some of the capture objects (e.g., at least some associated with at least one target antigen or one target antigen-antibody immunocomplex) may be spatially separated/segregated into a plurality of locations, and at least some of the locations may be addressed/interrogated. A measure of the concentration of tetraspanin antigen molecules or tetraspanin antigen-antibody immunocomplexes in the sample may be determined based on the information received when addressing the locations.

For example, in some cases, a measure of the concentration may be based at least in part on the number of locations (e.g., microwells) determined to contain a capture object that is or was associated with at least one detectable moiety. In some embodiments, such as digital immunoassays for detection of a tetraspanin antigen as described herein, the number of locations determined to contain a capture object that is or was associated with at least one detectable moiety may be related to the concentration of the target molecule in the sample. In other cases and/or under differing conditions, a measure of the concentration may be based at least in part on an intensity level of at least one signal indicative of the presence of a plurality of target molecules (e.g., tetraspanin antigen molecules or tetraspanin antigen-antibody immunocomplexes) and/or capture objects associated with a target molecule (e.g., tetraspanin antigen molecules or tetraspanin antigen-antibody immunocomplexes) at one or more of the addressed locations. In some embodiments, the number/fraction of locations containing a capture object but not containing a detectable moiety or a target molecule (e.g., tetraspanin antigen molecules or tetraspanin antigen-antibody immunocomplexes) may also be determined and/or the number/fraction of locations not containing any capture object may also be determined.

In some embodiments, multiple groups of distinguishably labeled capture objects or detectable moieties in complex with the capture objects may be detected simultaneously. Simultaneous addressing/detection can be accomplished by using various techniques, including optical techniques (e.g., using a charge coupled device (CCD) detector, charge-injection device (CID), or complementary-metal-oxide-semiconductor detector (CMOS) detector). Any suitable detector may be used in the methods described herein.

In some embodiments, the detection comprises detecting one or more antigen-antibody immunocomplexes bound to the plurality of capture objects.

In some embodiments, multiple detectable moieties on the detection probes bound to the one or more antigen-antibody immunocomplexes are detected on a single capture object.

In some embodiments, the multiple detectable moieties are detected by the intensity of the fluorescent signal of each of the capture objects.

In some embodiments, quantification of an intensity of the detectable moiety is proportional to a concentration of the tetraspanin antigen molecule or the tetraspanin antigen-antibody immunocomplex in the sample. In some embodiments, the concentration is extrapolated from a calibration curve generated by applying the methods herein to samples having known concentrations of the capture antigen or samples from healthy subjects. Comparing the intensity of the detectable labels, such a fluorescence, to the intensity generated by control samples may be used to distinguish between two different disease states.

Therefore, the methods of the disclosure provide highly sensitive and accurate measurement of concentrations of tetraspanin antigen or tetraspanin antigen-antibody immunocomplex. The methods allow for the detection of target molecules on the level of a single target molecule (e.g., tetraspanin antigen or tetraspanin antigen-antibody immunocomplex).

In some embodiments, the concentration of the tetraspanin antigen molecule is from about 0 to about 10 nanograms/milliliter (ng/mL). In some embodiments, the concentration of the tetraspanin antigen molecule is about 0 to about 0.2 ng/mL. In some embodiments, the concentration of the tetraspanin antigen molecule is about 0 to about 100 picograms/milliliter (pg/mL). In some embodiments, the concentration of the tetraspanin antigen molecule is about 0 to about 10 pg/mL. In some embodiments, the concentration of the tetraspanin antigen molecule is about 0 to about 1 pg/mL. In some embodiments, the concentration of the tetraspanin antigen molecule is about 0 to about 0.1 pg/mL.

In some embodiments, the concentration of the tetraspanin antigen-antibody immunocomplex is from about 0 to about 100 nanograms/milliliter (ng/mL).

EVs may be directly assayed from the biological samples, such that the level of EV is determined or the one or more biomarkers of the EVs are determined without prior isolation, purification, or concentration of the EVs. Alternatively, in some embodiments, an EV may be purified prior to analysis. EVs can be purified using one or more of the methods disclosed herein for purification of EVs.

Analysis of an EV can include quantitating the amount of one or more EV populations in a biological sample. For example, a heterogeneous population of EVs can be quantitated, or a homogeneous population of EVs, such as a population of EVs with a particular biomarker profile, or derived from a particular cell type (cell-of-origin specific EVs) can be purified from a heterogeneous population of exosomes and quantitated. Analysis of an exosome can also include detecting, quantitatively or qualitatively, a particular biomarker profile or a bio-signature, of an EV. An enriched population of EVs can be obtained from a biological sample derived from any cell or cells capable of producing and releasing EVs into the bodily fluid.

In some embodiments, more than one group of capture objects comprising capture moieties that bind to multiple epitopes on a tetraspanin antigen molecule is used in the methods disclosed herein. Simultaneous detection and quantification of the multiple epitopes on the antigen molecule can significantly increase the accuracy and sensitivity of the described methods.

### IV. Extracellular Vesicles

Extracellular vesicles (EVs) are a class of membrane bound organelles secreted by various cell types. By “extracellular vesicle” as provided herein is meant a cell-derived vesicle having a membrane that surrounds and encloses a central internal space. Membranes of EVs can be composed of a lipid bi-layer having an external surface and an internal surface bounding an enclosed volume. Such membranes can have one or more types of cargo, such as proteins, embedded therein. EVs include all membrane-bound vesicles that have a cross-sectional diameter smaller than the cell from which they are secreted. EVs can have a longest dimension, such as a longest cross-sectional dimension, such as a cross-sectional diameter ranging from 10 nm to 1000 nm, such as 20 nm to 1000 nm, such as 30 nm to 1000 nm, such as 10 to 100 nm, such as 20 to 100 nm, such as 30 to 100 nm, such as 40 to 100 nm, such as 10 to 200 nm, such as 20 to 200 nm, such as 30 to 200 nm, such as 40 to 200 nm, such as 10 to 120 nm, such as 20 to 120 nm, such as 30 to 120 nm, such as 40 to 120 nm, such as 10 to 300 nm, such as 20 to 300 nm, such as 30 to 300 nm, such as 40 to 300 nm, such as 50 to 1000 nm, such as 500 to 2000 nm, such as 100 to 500 nm, such as 500 to 1000 nm and such as 40 nm to 500 nm, each range inclusive.

EVs are important for intercellular communications within the human body and involved in many pathophysiological conditions such as cancer or neurodegenerative disease. EVs are abundant in various patient biological samples, e.g., biological fluids, including but not limited to blood, plasma, serum, cerebrospinal fluid, urine, saliva, breast milk, synovial, amniotic, and lymph fluids.

Membrane proteins can reflect the cellular environment the EV came from, for example a healthy or a tumor cell, or from a particular cell type, for example a specific breast cancer cell type. This genetic material can also hold clues to where the EV came from in the body, and how the EV may be interacting as a signaling messenger in the body.

The ability to detect EVs in various patient biological fluids has been shown to correlate well with disease progression, immune response, and toxicity; thus, the measurement and quantification of EVs could aid in disease diagnosis, and monitoring of treatment response. All disease states can have molecular signatures reflected in EVs. EVs can have molecular signatures reflected in, for example, but not limited to, cancer progression, cancer metastasis, melanoma, breast cancer, lung cancer, ovarian cancer, kidney cancer, glioblastoma, brain cancer, development of autoinflammatory disease such as Systemic lupus erythematosus, rheumatoid arthritis, multiple sclerosis, inflammatory bowel disease, neurodegenerative diseases such as Alzheimer's disease or Parkinson's disease, prion disease, transmissible spongiform encephalopathy, Creutzfeldt-Jakob disease, synucleinopathy, Dementia, multiple system atrophy, Huntington's disease, amyotrophic lateral sclerosis, leukemia, and more.

EVs as provided herein include exosomes. By “exosome” is meant a cell-derived vesicle composed of a membrane enclosing an internal space, wherein the vesicle is generated from a cell by fusion of the late endosome with the plasma membrane or by direct plasma membrane budding, and wherein the vesicle has a longest dimension, such as a longest cross-sectional dimension, such as a cross-sectional diameter, ranging for example, from 10 nm to 150 nm, such as 20 nm to 150 nm, such as 20 nm to 130 nm, such as 20 nm to 120 nm, such as 20 to 100 nm, such as 40 to 130 nm, such as 30 to 150 nm, such as 40 to 150 nm, or from 30 nm to 200 nm, such as 30 to 100 nm, such as 30 nm to 150 nm, such as 40 nm to 120 nm, such as 40 to 150 nm, such as 40 to 200 nm, such as 50 to 150 nm, such as 50 to 200 nm, such as 50 to 100 nm, or from 10 to 400 nm, such as 10 to 250 nm, such as 50 to 250 nm, such as 100 to 250 nm, such as 200 to 250 nm, such as 10 to 300 nm, such as 50 to 400 nm, such as 100 to 400 nm, such as 200 to 400 nm, each range inclusive. As used herein, “inclusive” refers to a provided range including each of the listed numbers. Unless noted otherwise herein, all provided ranges are inclusive.

An exosome is typically created intracellularly when a segment of the cell membrane spontaneously invaginates and is ultimately exocytosed. As used herein, exosomes can also include any shed membrane bound particle that is derived from either the plasma membrane or an internal membrane. Exosomes can also include cell-derived structures bounded by a lipid bilayer membrane arising from both herniated evagination (blebbing) separation and sealing of portions of the plasma membrane or from the export of any intracellular membrane-bounded vesicular structure containing various membrane-associated proteins, including surface-bound molecules derived from the host circulation that bind selectively to the exosomal proteins together with molecules contained in the exosome lumen, including but not limited to mRNAs, microRNAs or intracellular proteins. Blebs and blebbing are further described in Charras et al, Nature Reviews Molecular and Cell Biology, Vol. 9, No. 11, p. 730-736 (2008). Exosomes can also include membrane fragments.

### V. Samples

Any suitable sample may be used in the context of the present disclosure. For example, in some embodiments, the sample is a biological sample. In some embodiments, the sample is a sample obtained from a cell culture. In some embodiments, the sample is a liquid sample, e.g., a liquid biological sample. Exemplary liquid samples include, without limitation, body fluids, such as lymph, whole blood (including fresh or frozen), plasma (including fresh or frozen), serum (including fresh or frozen), a blood fraction containing peripheral blood mononuclear cells, urine, saliva, semen, sweat, lacrimal fluid, synovial fluid, cerebrospinal fluid, feces, mucous, vaginal fluid, and spinal fluid. Methods or obtaining tissue biopsies and body fluids from mammals are well known in the art.

The volume of the fluid sample analyzed may potentially be any amount within a wide range of volumes, depending on a number of factors such as, for example, the number of capture objects used/available, the number of detection probes, and the like. As non-limiting examples, the sample volume may be about 0.01 μl, about 0.1 μl, about 1 μl, about 5 μl, about 10 μl, about 100 μl, about 1 mL, about 5 mL, about 10 mL, or the like. In some cases, the volume of the fluid sample is between about 0.01 μl and about 10 mL, between about 0.01 μl and about 1 mL, between about 0.01 μl and about 1.5 mL, between about 0.01 μl and about 2 mL, between about 0.01 μl and about 10 mL, between about 0.01 μl and about 100 μl, or between about 0.1 μl and about 10 μl. In some embodiments, each sample is tested in duplicates or triplicates. In some embodiments, the sample volume is about 25 μl per replicate. In some embodiments, the sample volume is about 10 μl per replicate.

In some embodiments, the fluid sample may be diluted prior to use in a method described herein. For example, in embodiments where the source of a target molecule is a body fluid (e.g., blood, plasma, or serum), the fluid may be diluted with an appropriate solvent (e.g., a buffer such as PBS buffer). A fluid sample may be diluted about 1-fold, about 2-fold, about 3-fold, about 4-fold, about 5-fold, about 6-fold, about 10-fold, about 50-fold, about 100-fold, or greater, prior to use.

The sample may be added to a solution comprising a plurality of capture objects or detectable moieties, or the plurality of capture objects or detectable moieties may be added directly to or as a solution to the sample. The sample may be also be added to a solution comprising a size-exclusion beads, or a plurality of size-exclusion beads may be added directly to sample. In some embodiments, the size-exclusion beads are combined with a buffer, e.g., PBS, to create a slurry, and the sample is combined with the slurry.

This disclosure is further illustrated by the following examples which should not be construed as limiting. The contents of all references, patents and published patent applications cited throughout this application, as well as the Figures, are hereby incorporated by reference.

### EXAMPLES

### Example 1: Methods for Quantifying and Purifying Extracellular Vesicles

The following Materials and Methods were used in the Example below.

### Materials and Methods

### Human Sample Handling

Pre-aliquoted pooled human cerebrospinal fluid (CSF) or plasma were ordered from BioIVT. These same two pools were used for all main figures throughout the paper in order to ensure comparable analysis of methods. For all EV purification technique comparisons one 0.5 mL sample was used for each purification method. CSF or plasma was thawed at room temperature. Immediately after the sample had thawed, 100× Protease/Phosphatase Inhibitor Cocktail™ was added (Cell Signaling Technology #5872S) was added to 1×. The sample was then centrifuged at 2000×g for 10 minutes. Next, the supernatant was centrifuged through a 0.45 μM Corning Costar SPIN-X™ centrifuge tube filter (Sigma-Aldrich) at 2000×g for 10 minutes to remove any remaining cells or cell debris, and further analysis proceeded.

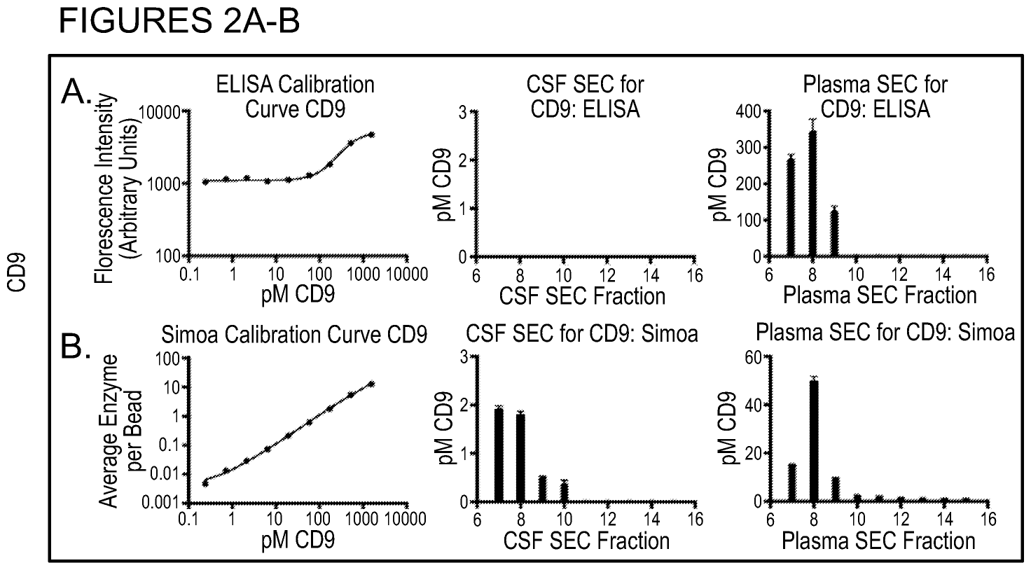

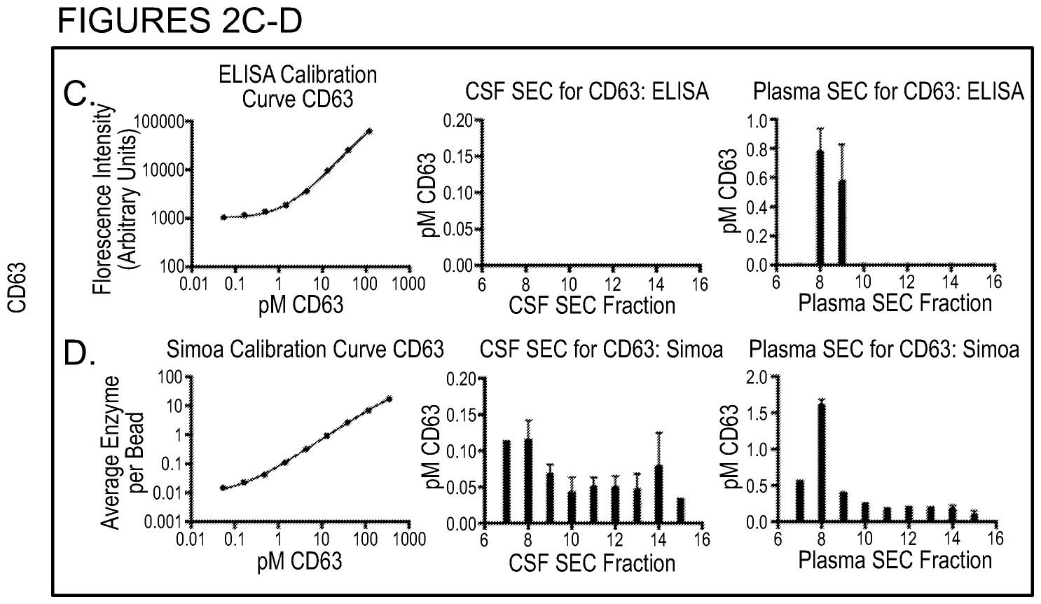

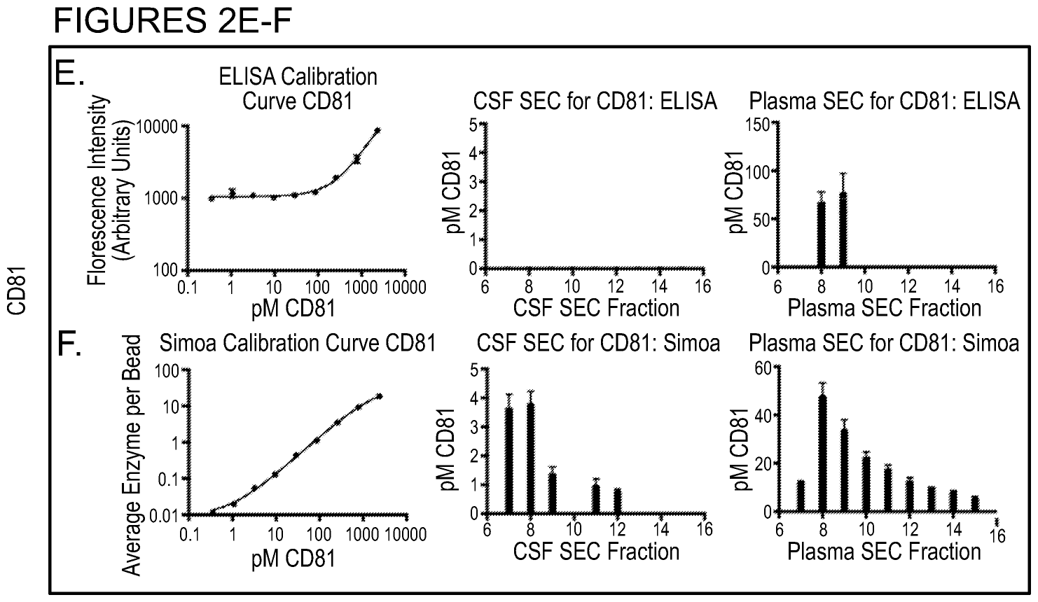

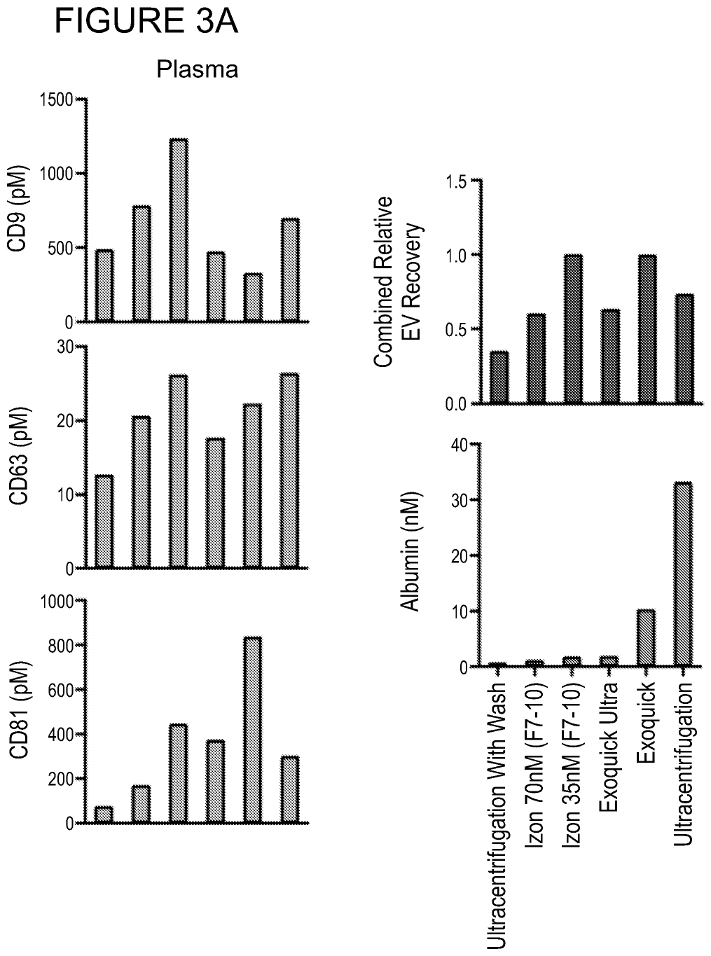

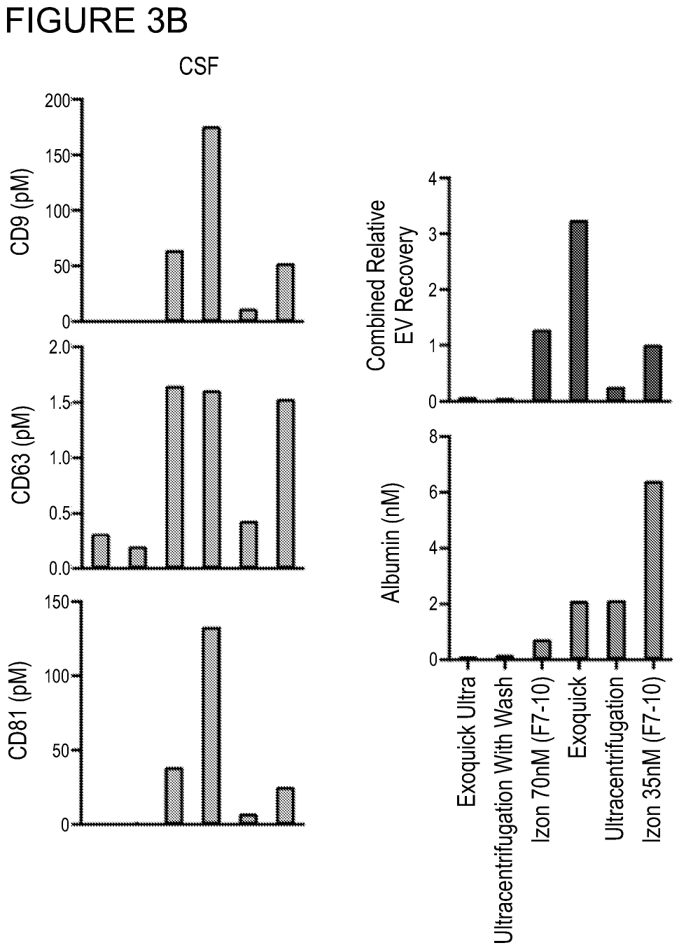

To obtain the results depicted in FIG. 2, one 0.5 mL sample was fractionated by SEC (Izon35 nM) and the fractions were divided evenly to be used in each of the three techniques (Simoa®, ELISA, and Western). For all EV purification technique comparisons (results depicted in FIGS. 3-6), one 0.5 mL sample was used for each purification method.

### Simoa® Assays

Candidate capture antibodies were coupled to Carboxylated Paramagnetic Beads from the Simoa® Homebrew Assay Development Kit™ (Quanterix) using EDC chemistry (Thermo Fisher Scientific). Candidate detection antibodies were conjugated to biotin using EZ-Link NHS-PEG4 Biotin (Thermo Fisher Scientific). Reagents were cross-tested for signal against the following recombinant proteins for CD9, CD63, CD81, and albumin: ab152262 (Abcam), TP301733 (Origene), CD81 Origene TP317508 (Origene), ab201876 (Abcam) on a Simoa® HD-X Analyzer (Quanterix). The antibody pairs that gave the highest signal to background ratio were further validated in three ways. First, plasma and CSF were serially diluted in sample buffer in order to demonstrate endogenous dilution linearity. Next, the recombinant protein used in the calibration curve was added to CSF and plasma at two different concentrations in order determine that spike and recovery was within 70-130%. For comparison of ELISA, Simoa® and Western, samples were hand diluted. For all other figures on-board dilution was performed with 4× dilution of each tetraspanins, while 20× hand dilution was used for albumin.

### Western Blotting

Western blotting was performed as previously described in detail (26). Equal volumes of each SEC fraction were loaded on a Bolt™ 4-12% Bis-Tris Plus gel (Thermo Fisher Scientific) after addition of 4× Bolt LDS™ sample buffer (Thermo Fisher Scientific) and then transferred onto a nitrocellulose membrane using the iBlot2 Dry Blotting System™ (Thermo Fisher Scientific). The following primary antibodies were used for western blot at the corresponding dilutions in milk overnight: MM2/57 for CD9 (Millipore Sigma) at 1:1000, h5c6 for CD63 (BD Biosciences) at 1:1000. M38 for CD81 (Thermo Fisher Scientific) at 1:666. After three washes with PBST, anti-mouse IgG Secondary antibody (Bethyl Laboratories) was added for 2 hours in milk buffer at 1:2000 dilution. After three more washes in PBST, SpectraQuant HRP-CL Spray Chemilumenescent Detection Reagent™ (BridgePath Scientific) was used to develop westerns imaging was performed on the Sapphire Biomolecular Imager™ (Azure Biosystems).

### Plate-Based ELISA

Simoa® assays were transferred to a plate-based ELISA for comparison. Capture antibody was diluted in ELISA Coating Buffer (BioLegend) at a concentration of 4 μg/mL and 100 μL was coated per well on a Nunc MaxiSorp™ ELISA plate (BioLegend). Plates were incubated with capture antibody overnight at 4° C. Subsequently, the plate was washed 3× with 200 μL PBST. Sample was added to each well and incubated at room temperature for 3 hours. The plate was then washed 3× with 200 μL PBST and 100 μL of corresponding detection antibody was incubated for 1 hour. Detection antibody was then removed and the plate was washed 3× with 200 μL PBST. 1004, Streptavidin labeled β-galactosidase from the Simoa® Homebrew Assay Development Kit (Quanterix) was then added and incubated for 30 minutes. The plate was then washed 5× with 200 μL PBST and incubated with 100 μL of Resorufin β-D-Galactopyranoside, also from the Simoa® Homebrew Assay Development Kit (Quanterix), for 20 minutes in the dark. Plates were then imaged with a Tecan™ Plate Reader using Magellan v 7.2™ software at 555 nM excitation and 605 nM Emission.

### Preparation of Custom Sepharose™ Columns

The resins Sepharose™ CL-2B, Sepharose™ CL-4B, and Sepharose™ CL-6B (all from GE Healthcare) were washed in PBS. Briefly, the volume of resin was washed with an equal volume of PBS in a glass jar and then placed at 4° C. in order to let the resin settle completely (several hours or overnight). The PBS was then poured off and new PBS was again added for a total of 3 washes. Columns were prepared fresh on the day of use. Washed resin was poured into an Econo-Pac™ Chromotography column (Bio-Rad) at either 10 mL or 20 mL bed volume. The column was allowed to drip out until the column was solid at which time the top frit was placed securely at the top of the resin but without compression. PBS was then added at 1 mL above the frit until sample was ready to be added.

### Collection of Size Exclusion Chromatography Fractions

Once prepared, all columns were washed with at least 20 mL of PBS in the column. When sample was ready to be loaded, the column was allowed to fully drip out and, after last drop, CSF or plasma was added to the column. Immediately thereafter, 0.5 mL fractions were collected. As soon as the CSF or plasma completely went through the frit, PBS was added on top 1 mL at a time. For Izon and 10 mL columns fractions 6-21 were collected while for 20 mL columns fractions 12-27 were collected. For comparison of Simoa®, ELISA, and Western Blot, one 0.5 mL sample was fractionated by SEC using a qEVoriginal™ 35 nM column (Izon) into 0.5 mL fractions. Each of the fractions was then divided evenly to be used in each of the three techniques.

### Ultracentrifugation

Samples of filtered 0.5 mL plasma or CSF were added to 3.5 mL Open-Top Thickwall Polycarbonate™ ultracentrifuge tubes (Beckman Coulter) and PBS was added to fill tubes to the top. Samples were ultracentrifuged at 120,000 g for 90 minutes at 4 C in an Optima XPN-80™ ultracentrifuge (Beckman Coulter) using a SW55 Ti swinging-bucket rotor (Beckman Coulter). Afterwards, all supernatant was aspirated. Pellets were resuspended in PBS and transferred to 1.5 mL tubes for the “Ultracentrifuge” condition. For the “Ultracentrifuge with wash” condition, the ultracentrifuge tubes were filled to the top with PBS and samples were ultracentrifuged again at 120,000 g for 90 minutes. Supernatant was then aspirated and pellets were resuspended in 500 uL PBS.

### ExoQuick™ & ExoQuick ULTRA™

Samples of plasma or CSF were mixed with ExoQuick Exosome Precipitation Solution™ (System Biosciences) or ExoQuick ULTRA EV Isolation Kit™ for Serum and Plasma (System Biosciences) and protocols were performed according to manufacturer's instructions. For ExoQuick™, 0.5 mL of plasma or CSF was mixed with 126 uL of ExoQuick™ and incubated at 4 C for 30 minutes, followed by centrifugation at 1500 g for 30 minutes. Supernatant was removed and samples were centrifuged at 1500 g for an additional 5 minutes. Residual supernatant was removed and pellets were resuspended in 500 uL PBS. For Exoquick ULTRA™, 250 uL of plasma or CSF was used in accordance with instructions, and Simoa® values were corrected by multiplying by two to match 0.5 mL volume used for other samples. For each sample, 500 uL of EVs was eluted per column.

### EV Method Development Using Capto™ Core 700

Capto™ Core 700 (GE Healthcare) was pelleted at 800 g for 10 minutes and re-suspended in an equal volume of PBS to make a 50% slurry. Different quantities of slurry were added to 1 mL CSF in a 2 mL Eppendorf Tubes™ and mixed for 45 minutes at room temperature. Each tube was centrifuged at 800 g for 10 minutes, and the supernatant was transferred to a new tube and filtered through a 0.45 micron spin filter at 2000 g for 10 minutes.

### Results

### EV Analysis Using Single Molecule Arrays (Simoa®)

A new platform for EV quantification was developed using single molecule array (Simoa®) technology. Simoa® is generally used to quantify free proteins, but the inventors applied Simoa® to EVs by immuno-isolating single vesicles into microwell arrays with antibodies to transmembrane proteins on their surface (FIGS. 1A-D). As in conventional ELISA, Simoa® uses two antibodies: a capture antibody conjugated to a magnetic bead, and a detector antibody conjugated to an enzyme. But unlike in traditional ELISA, individual immuno-complexes are isolated into femtoliter wells that fit only one bead per well. In a given reaction, there are many more antibody-bound beads than target proteins, and therefore Poisson statistics dictate that only a single immuno-complex is present per well. This allows for counting “on wells” as individual proteins, or, in the case of EVs, antigen-positive vesicles. Thus, Simoa® can count single antigen-positive EVs with a specific transmembrane protein or combination of transmembrane proteins. The ratio of “on wells” to “off wells” can then be mapped against a standard calibration curve of recombinant protein of known concentration.

### Development of Simoa® Assays for Tetraspanins and Albumin

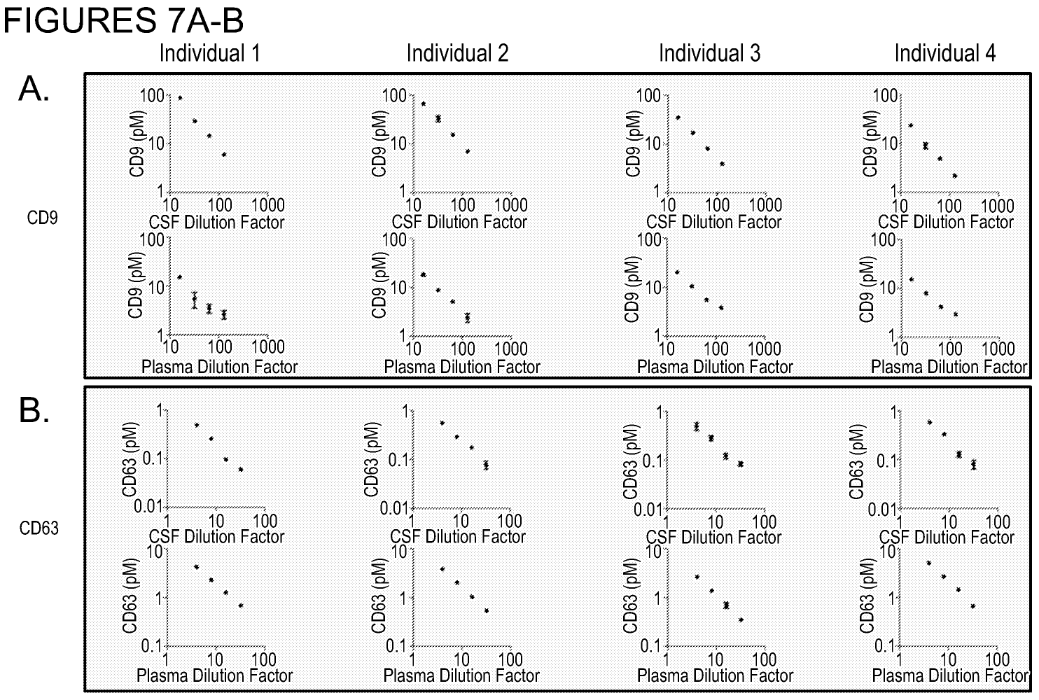

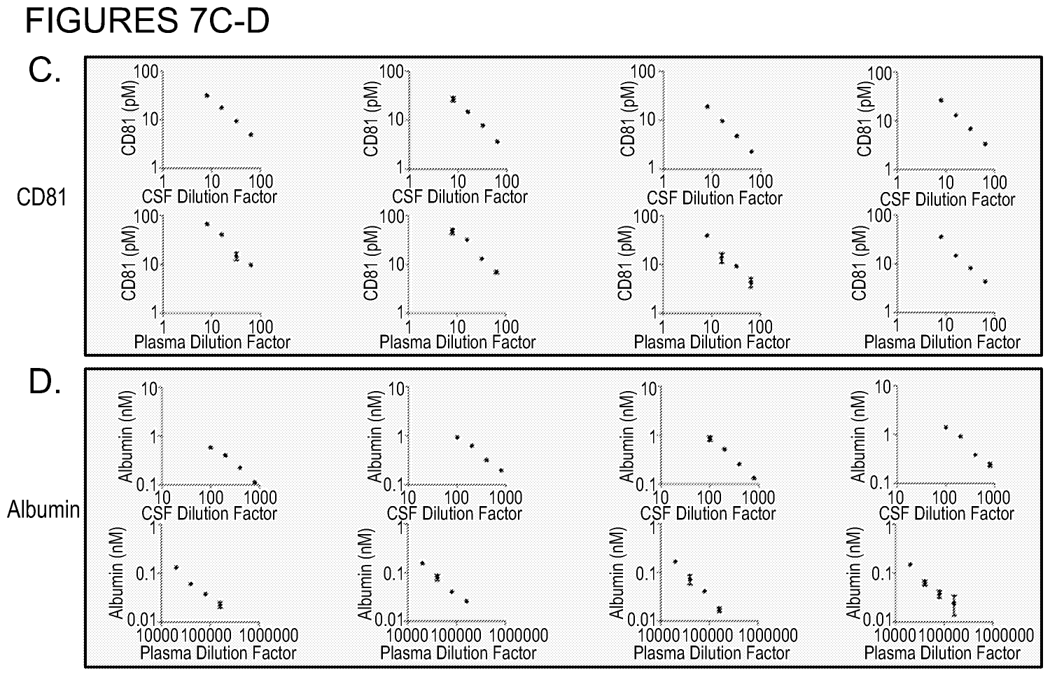

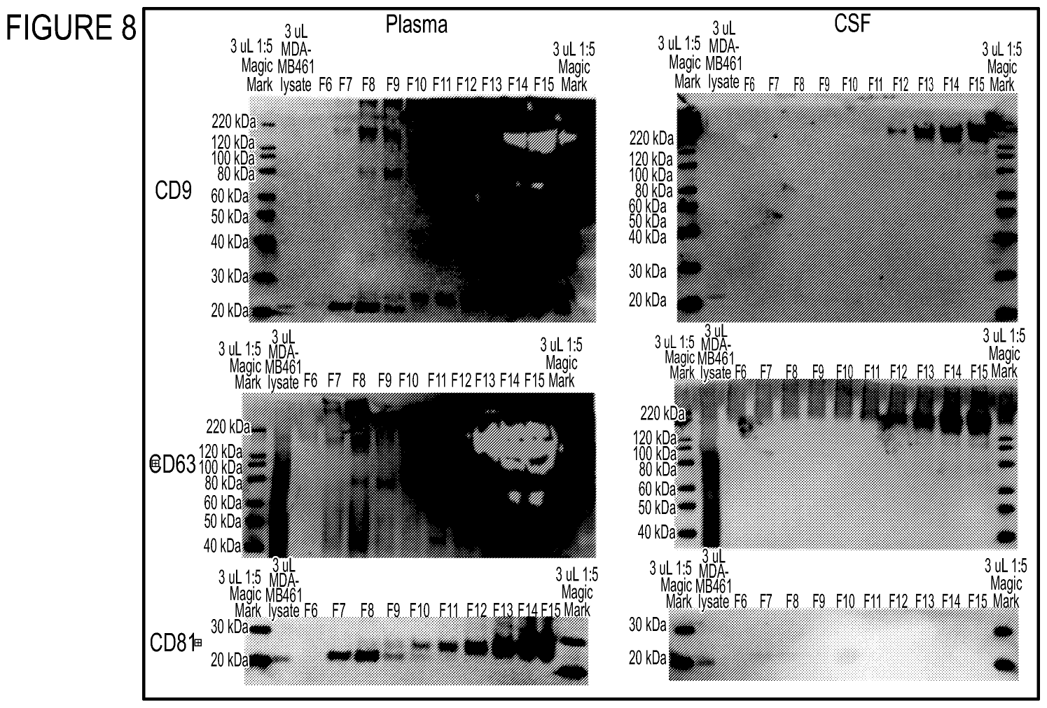

Assays were developed which measured the transmembrane proteins CD9, CD63 and CD81, as well as albumin in order to assess contamination of free proteins. To validate that the quantification was accurate and precise, three types of validation experiments were performed. First, each assay demonstrated endogenous dilution linearity (parallelism) in human CSF and plasma (see FIG. 7). Next, the respective recombinant protein used for the calibration curve was spiked into human CSF and plasma and the percent recovery was quantified. Each assay recovered between 70-130% of the spiked concentration indicating good assay precision (Table 2). Finally, it was demonstrated that the peak concentration of each protein after size exclusion chromatography of human plasma with an Izon 35 nM column matched between the Simoa® assay and the Western blot, in which size information could be used to demonstrate target validity (see FIGS. 2 and 8).

TABLE 2
Spike and Recovery of Simoa ® Assays in CSF and Plasma

| | | Average | | Average |
|---|---|---|---|---|
| | | Recovery | Plasma | Recovery |
| | CSF Dilution | (4 indi- | Dilution | (4 indi- |
| Protein | Factor/Spike | viduals) | Factor/Spike | viduals) |
| CD9 | 32x + 500 | 85% | 32x + 500 | 79% |
| | 32x + 1000 | 101% | 32x + 1000 | 74% |
| CD63 | 16x + 10 | 106% | 16x + 10 | 83% |
| | 16x + 50 | 105% | 16x + 50 | 74% |
| CD81 | 32x + 500 | 104% | 32x + 500 | 106% |
| | 32x + 1000 | 101% | 32x + 1000 | 94% |
| Albumin | 400x + 50000 | 90% | 80000x + 50000 | 85% |
| | 400x + 100000 | 85% | 80000x + 100000 | 90% |

Comparison of Simoa® Assay with Conventional Techniques

The sensitivity of the tetraspanin assays described herein and their ability to quantify CSF and plasma EVs was compared to standard methods such as Western blot and ELISA. It was found that the LOD of Simoa® assays were one to two orders of magnitude more sensitive than their respective standard ELISA. To ascertain whether this improved sensitivity was necessary for quantification of EVs in biological samples, human CSF and plasma were fractionated using a commercially available SEC column (Izon qEVoriginal 35 nM), and fractions were evenly split for downstream analysis using Simoa®, ELISA and Western Blotting.

Simoa® was able to quantify tetraspanins in EVs fractions 7-10 (the fractions where EVs are expected) for all markers in both CSF and plasma (FIG. 2B, D, F). ELISA was able to fully quantify fractions 7-10 only for plasma CD9. All other ELISAs gave readings for only a portion of the fractions while CD81 in CSF was completely undetectable (FIG. 2A, C, E). When the SEC fractions were analyzed by western blot, very high background was observed in the plasma fractions (from abundance of free proteins), but the tetraspanins were detectable and matched the general pattern of the Simoa® results. None of the tetraspanins were detected in any CSF fractions, however (FIG. 8). Thus, the only method that allowed quantification of EVs in SEC fractions for plasma and CSF was Simoa®.

### Application of Simoa® Comparison of Existing EV Purification Methods

In order to use EVs for diagnostic applications, the first step is to purify the sample of free protein and purify EVs for downstream analysis. Several techniques have been used to purify EVs for diagnostic applications and the degree of recovery and purity vary drastically amongst these methods. To date, a quantitative analysis of yield and purity has not been possible to due to a lack of techniques that distinguish EVs from similarly sized protein aggregates.

EV purification from plasma and CSF was directly compared with two commercially available SEC columns (Izon qEVoriginal 35 nM and 70 nM), two commercial precipitation kits (ExoQuick and ExoQuick ULTRA), and ultracentrifugation with or without a wash step. For each method, we used samples of human plasma or CSF that were pooled and aliquoted, allowing direct comparison of the different methods. To differentiate EVs from cells, cell debris, or large vesicles, all samples were first centrifuged and then filtered through 0.45-micron filter (FIG. 1).

With Simoa assays, the inventors assessed the quantity of each tetraspanin as well as albumin contamination across a variety of methods in both plasma and CSF (FIGS. 3A and B). By comparing the ratio of the combined tetraspanins to albumin the methods which gave the highest recovery of EVs and purity from contaminating proteins were identified. Normalizing all measurements to the total protein detected across fractions 7-10 in the Izon qEVoriginal 35 nM SEC column and averaging the recovery of the 3 tetraspanins the method with the highest recovery and highest purity for each biofluid was identified. In plasma, the Izon qEVoriginal 35 nM SEC column (collecting fractions 7-10) yielded both the highest recovery of EVs and the highest purity of EVs when comparing EV yield to albumin contamination (FIG. 3A). In CSF, ExoQuick yielded the highest level of EVs while Izon qEVoriginal 70 nM yielded the highest purity (FIG. 3B).

### Optimization of SEC for Extracellular Vesicle Purification in Plasma and CSF

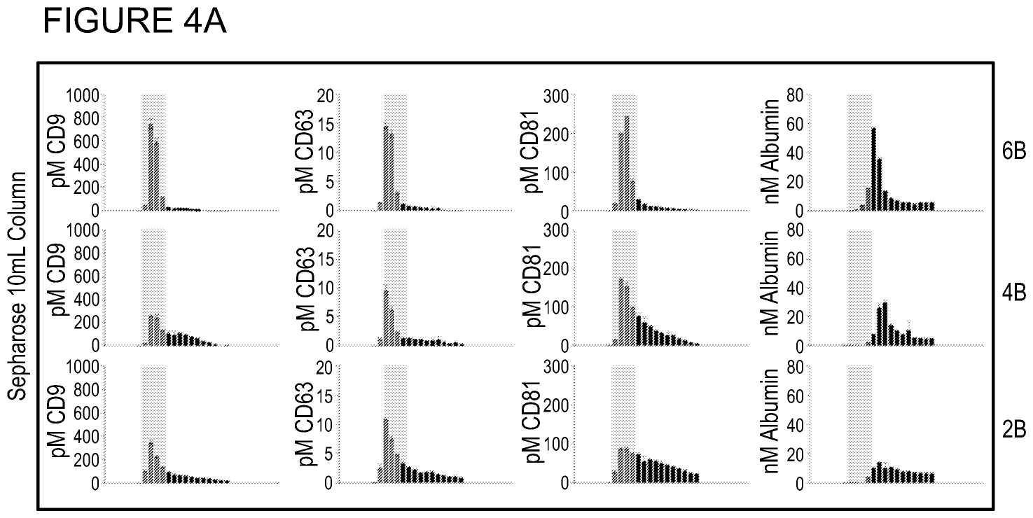

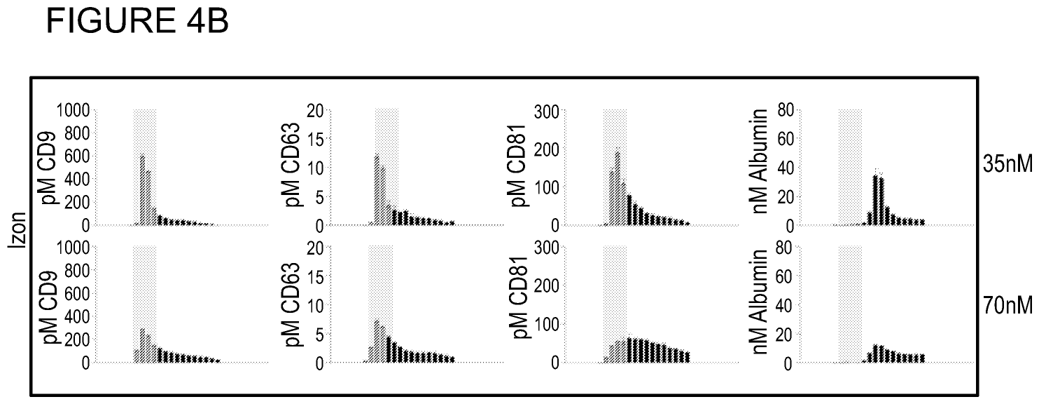

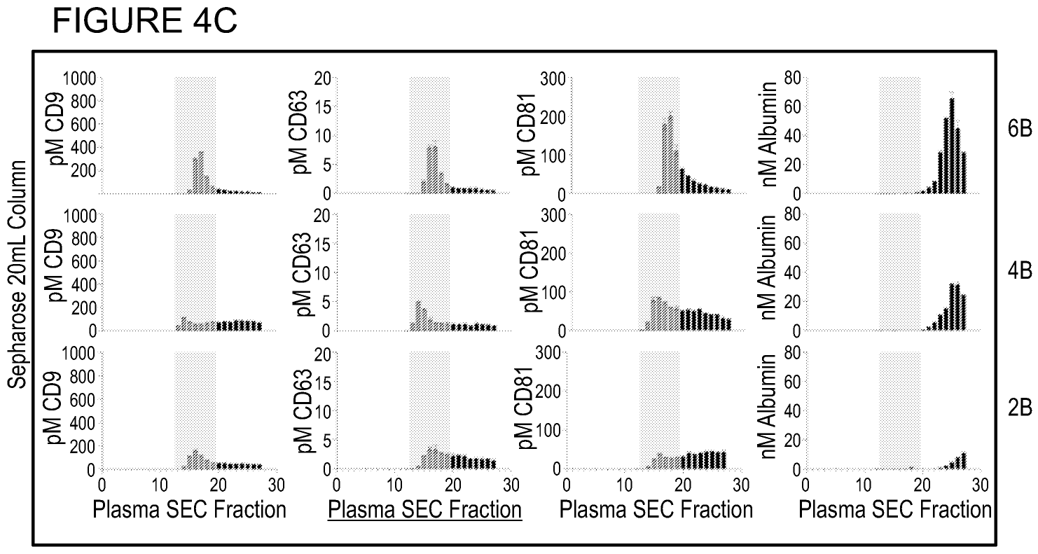

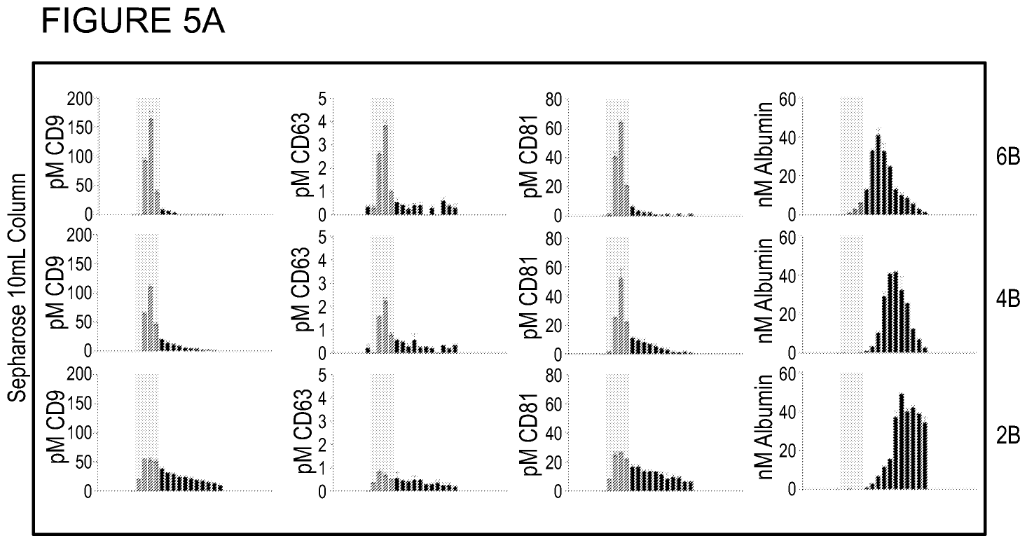

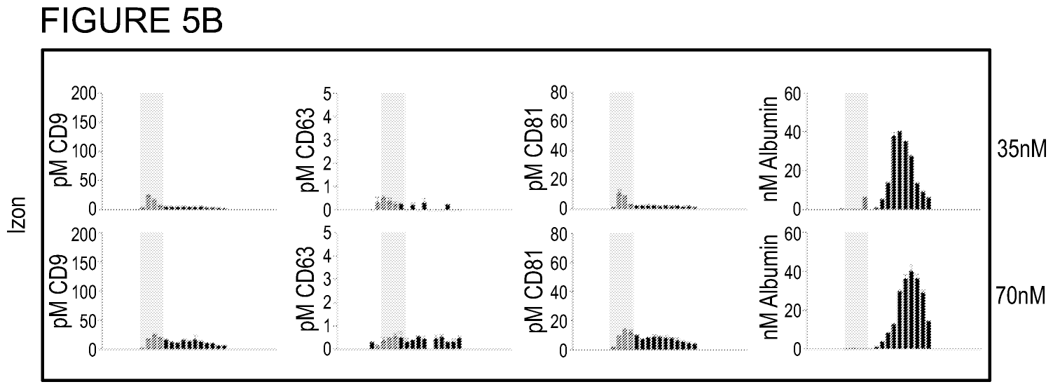

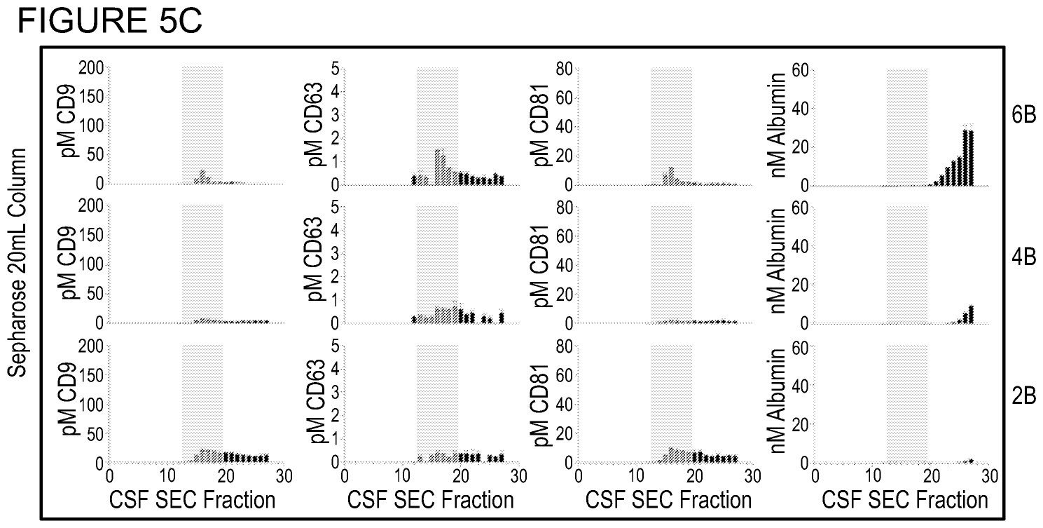

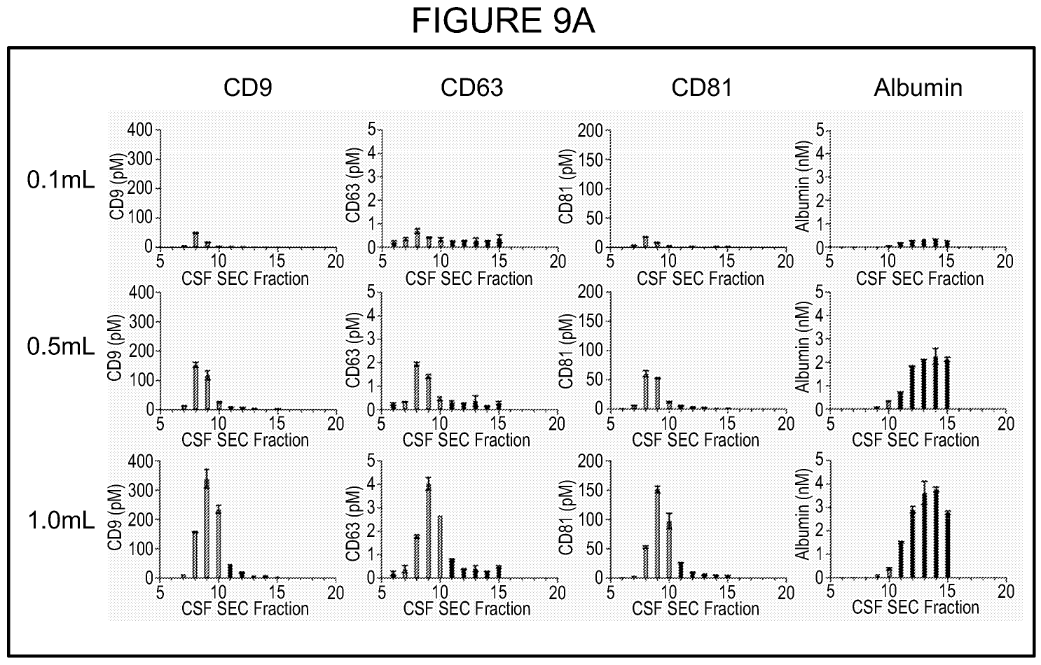

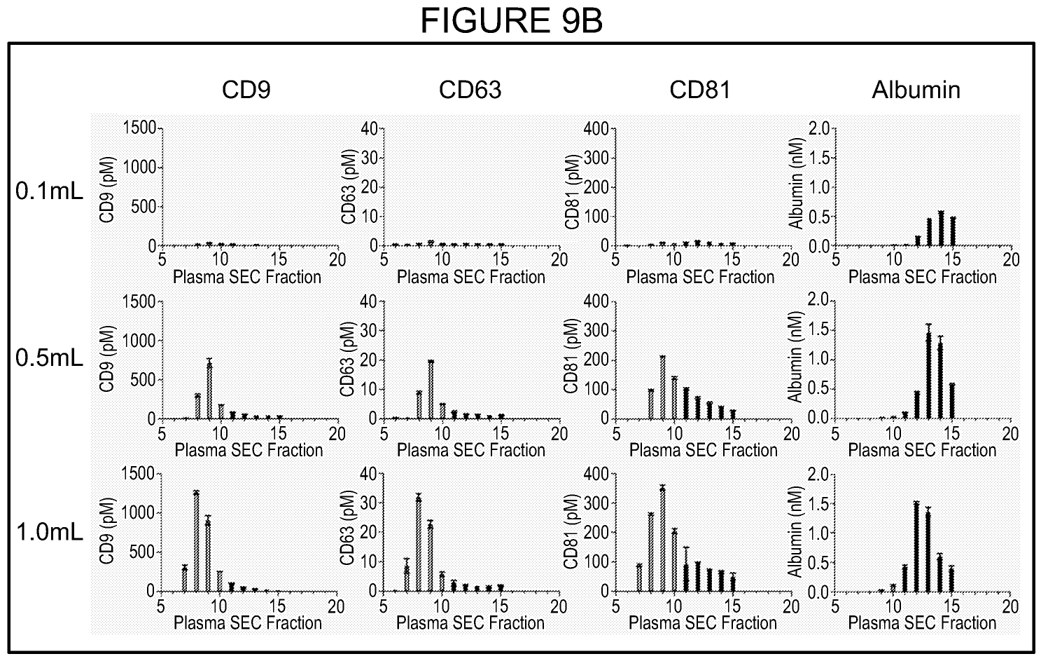

The effect of several parameters on EV purification from 0.5 mL samples of plasma and CSF using SEC was tested using Simoa®. SEC columns were developed that improve EV yield and purity beyond that of the commercially available columns. To do so, three Sepharose™ resins (CL-2B, CL-4B and CL-6B) at two column heights (10 and 20 mL) were tested. This comprehensive comparison led to several conclusions. First, it was found that resins with smaller pore sizes led to higher yield. Sepharose™ CL-6B, which has the smallest pore size, gave the highest yield of EVs, although at a cost of higher albumin contamination. For all SEC columns, higher purity could also be achieved by taking a smaller number of the fractions (e.g., 7-9 instead of 7-10), albeit with lower yield. This difference was quantified for all columns (see FIGS. 4 and 5). It was also found that doubling the height of any given column from 10 to 20 mL resulted in better separation between EVs and free proteins, leading to higher purity but lower EV recovery (see FIGS. 4 and 5). When different volumes of plasma and CSF were compared for a Sepharose™ CL-6B 10 mL column, it was found that volume loaded had an effect on purity, with larger loading volumes leading to lower purity (see FIGS. 9A-B).

### Direct Comparison of all EV Purification Methods

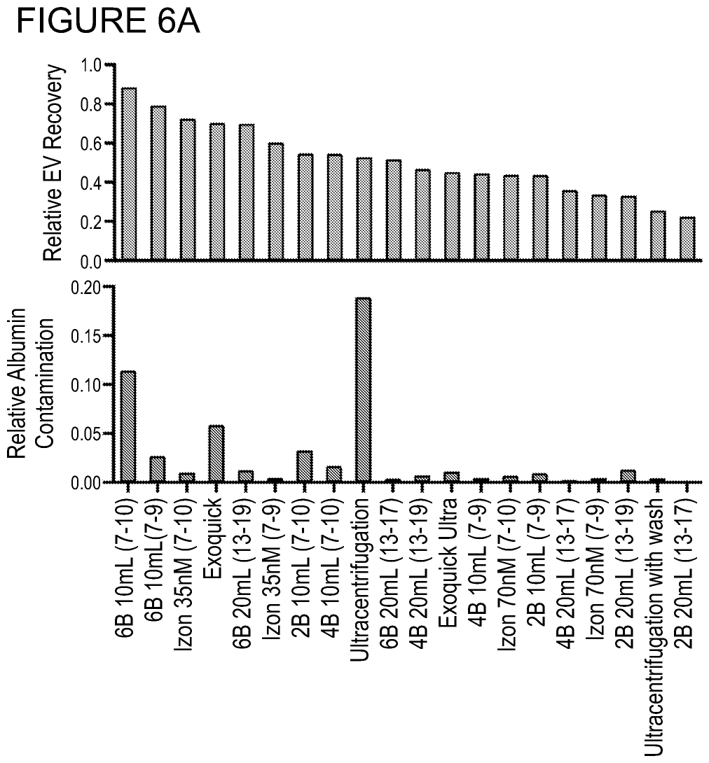

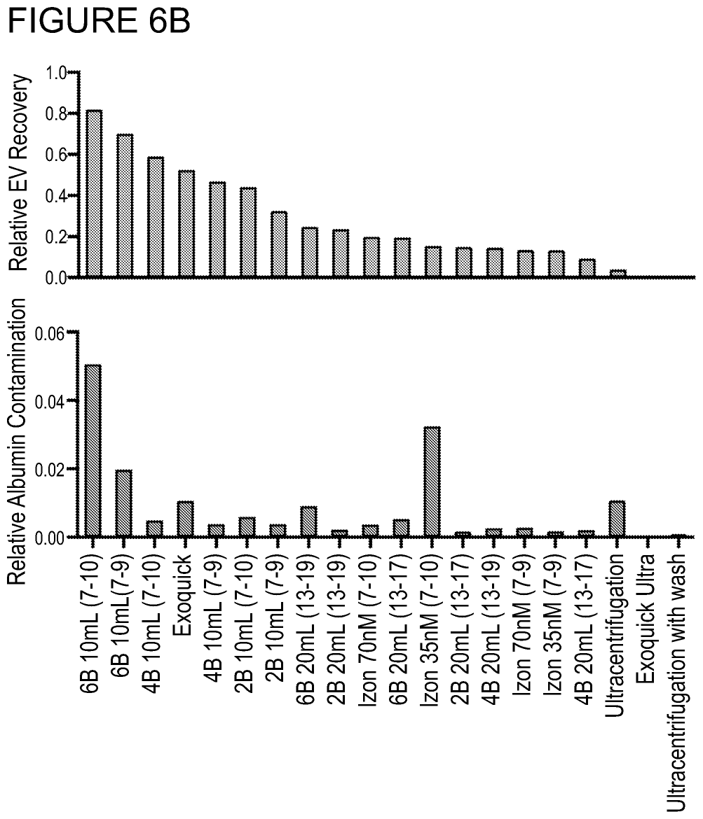

Combining all of the Simoa data, the relative yield and purity across methods was compared. Since Sepharose™ CL-6B 10 mL columns gave the highest yield in both plasma and CSF, the sum of each marker across all fractions in that condition was used to normalize to. Percent recovery was calculated by adding the sum of each of the three tetraspanins for each purification method and dividing by the sum of the tetraspanins in all of the different fractions in Sepharose™ CL-6B 10 mL (see FIGS. 6A-B). Percent albumin was calculated by taking the amount of albumin in each purification relative to the sum of albumin detected in all of the fractions of Sepharose™ CL-6B 10 mL.

In plasma, the other techniques that demonstrated the highest recovery (>70% relative to Sepharose™ CL-6B 10 mL) were Sepharose™ CL-6B 20 mL column, Izon qEV original 35 nM column, and ExoQuick (see FIG. 6A). Although the highest recovery of EVs was found in the Sepharose™ CL-6B 10 mL column, the highest purity of the three columns with the highest yield was achieved with the Izon qEVoriginal 35 nM column.

In CSF, the Izon qEVoriginal 35 nM columns performed worse than several other techniques. The highest purity technique in CSF of the three columns with the highest yield was the Sepharose™ CL-4B 10 mL column, yielding more than 50% of the EVs found in Sepharose™ CL-6B 10 mL but several hundred fold less albumin (see FIG. 6B). These findings indicate that the best purification method depends both on the biofluid of interest downstream application since there is a tradeoff between EV yield and purity.

### High-Throughput “In-Slurry” Method for Extracellular Vesicle Purification

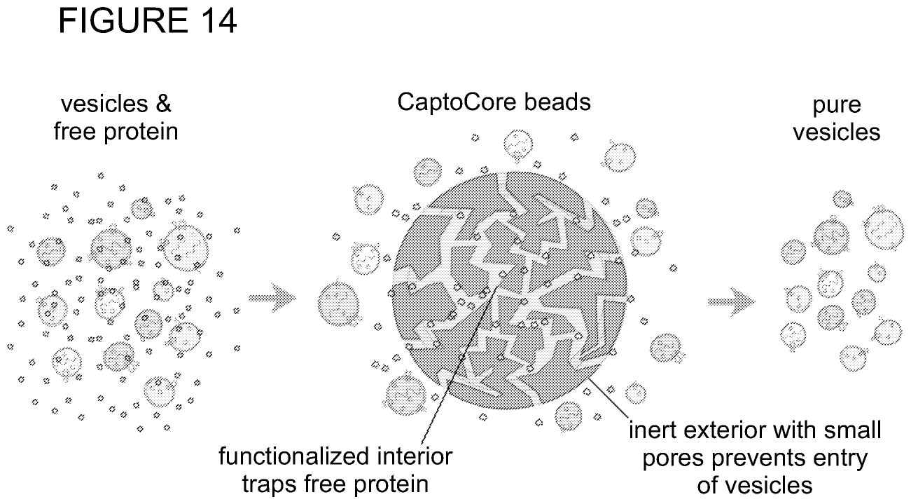

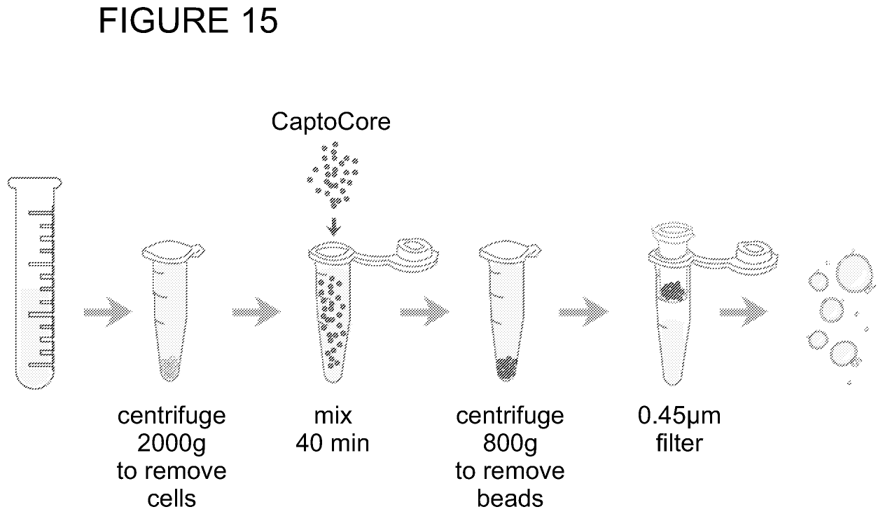

A new, high-throughput method was developed to purify EVs from biofluids. Using Capto™ Core 700, which is a bead containing an octylamine ligand, molecules smaller than 700 kDa are trapped inside the beads. As EVs are larger than this cutoff, the beads effectively remove free proteins from the biofluid. The beads can then be removed and discarded using conventional centrifugation, leaving a pure solution of EVs (FIGS. 14 and 15).

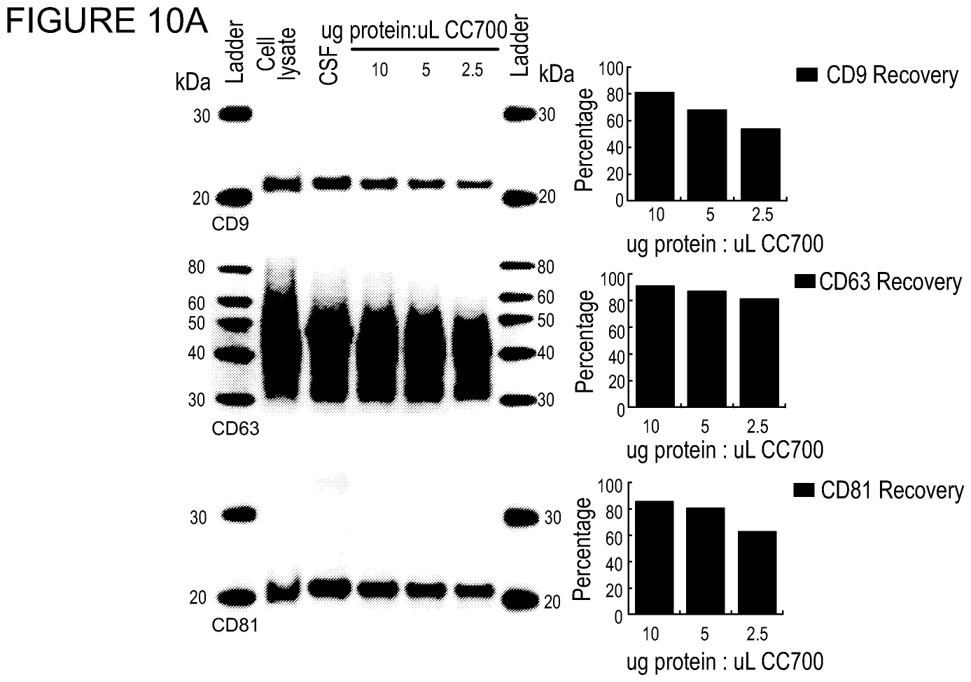

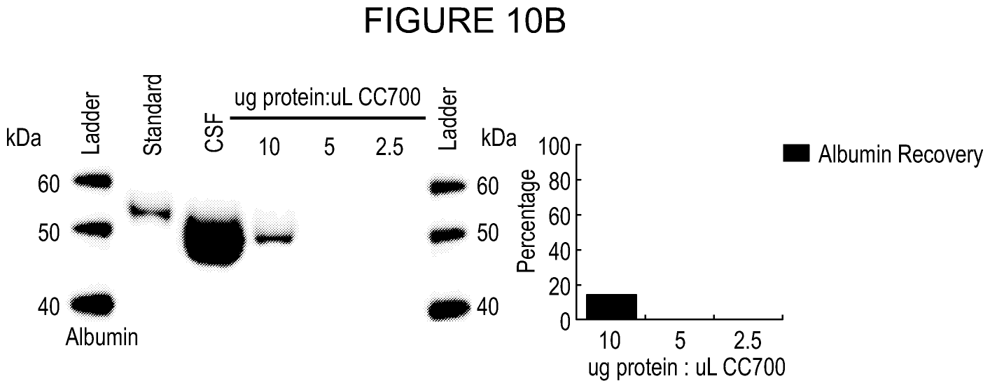

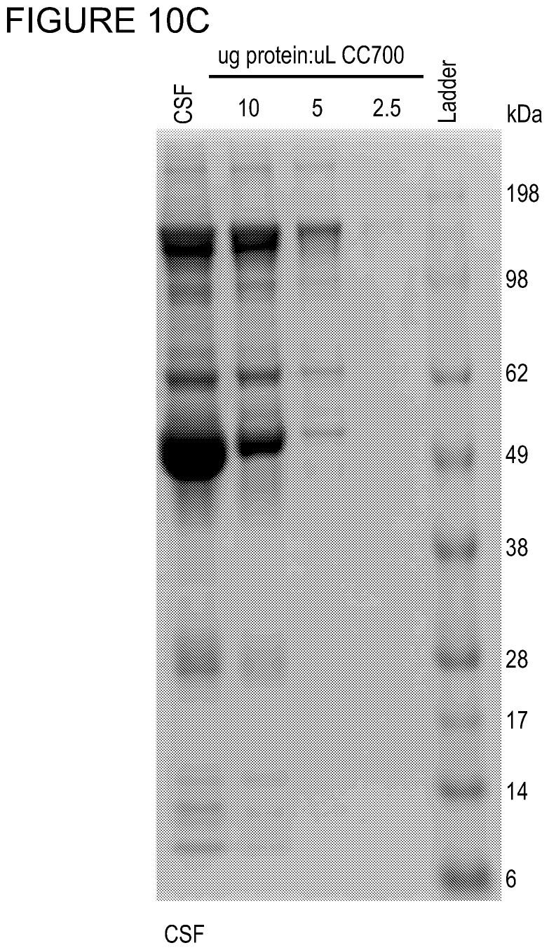

Different concentrations of Capto™ Core 700 Resin were compared in purifying EVs from 1 mL human CSF. Different amounts of Capto™ Core 700 resin were added to the CSF sample and compared. The ratio between the Capto™ Core 700 resin and the total protein was about 10 μg protein per 1 μL resin, about 5 μg protein per 1 μL resin, and about 2.5 protein per 1 μL resin. As shown in FIGS. 10A-C, an increasing purity for EV markers (CD9, CD63 and CD81) was observed (FIG. 10A) after increasing amounts of Capto™ Core 700 resin, as measured by a depletion of a major contaminating protein, albumin, by Western Blot (FIG. 10B) and total protein by Ponceau Stain (FIG. 10C). It was found that most EVs can still be recovered even at the highest quantity of resin by Western Blot or Simoa®.

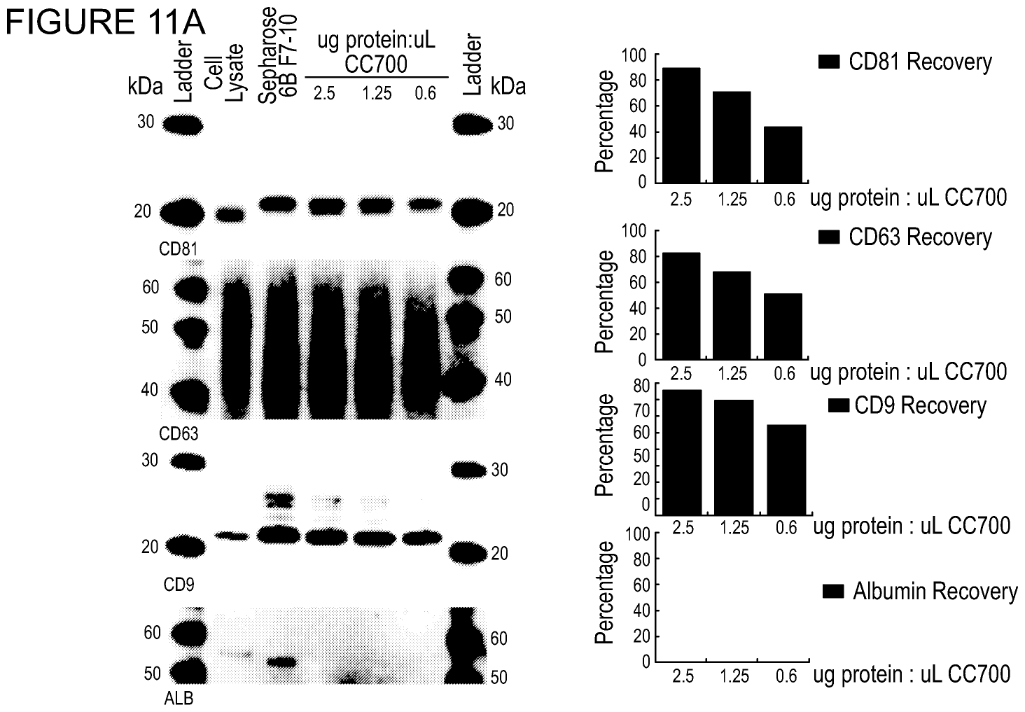

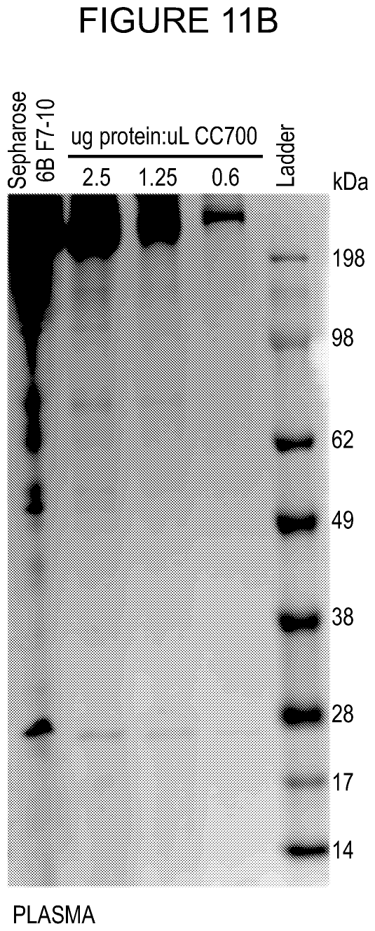

Capto™ Core 700 resin was also used in combination with Sepharose™ CL-6B 10 mL columns for the purification of EVs from human plasma samples. Plasma samples were first subjected to a Sepharose™ CL-6B 10 mL column. Fractions 7-10 were collected and added with increasing amounts of Capto™ Core 700 resin. Since plasma is a protein heavy sample, the ratio between the Capto™ Core 700 resin and the total protein was higher, for example, from about 2.5 μg protein per 1 μL resin, 1.25 μg protein per 1 μL resin, to 0.6 protein per 1 μL resin. As shown in FIGS. 11A-B, an increasing purity for EV markers (CD9, CD63 and CD81) was observed after increasing amounts of Capto™ Core 700 resin added to the Sepharose™ CL-6B 10 mL column fractions 7-10. A complete removal of albumin was observed even at a ratio of 2.5 μg protein per 1 μL Capto™ Core 700 resin, and a significant reduction in total protein level was also observed after the step of Capto™ Core 700 resin.

### Dual Mode Chromatography Method for Extracellular Vesicle Purification

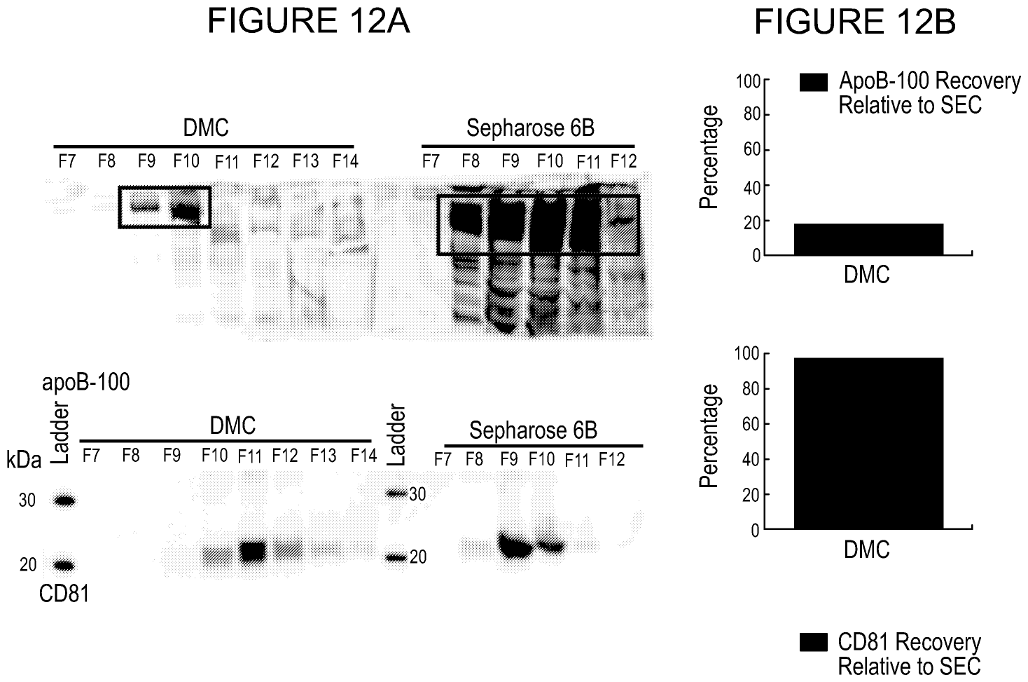

A combination of chromatography steps was developed to purify EVs from biofluid, e.g., plasma, in order to further reduce potential contaminants, such as lipoproteins, e.g., ApoB100. A dual mode chromatography was developed by layering 10 mL of a Sepharose™ CL-6B resin on top of 2 mL of a cation exchange resin (Fractogel® EMD-SO₃⁻ resin). Human plasma samples were added the dual mode chromatography. Fractions 7-14 were collected for Western blot analysis for both the contaminating lipoprotein component ApoB100, and the EV marker CD81. A significant reduction in the level of ApoB100 was observed in samples collected from the dual mode chromatography, as compared to the samples collected from the Sepharose™ CL-6B resin alone (FIGS. 12A and 12B). In contrast, the percentage recovery for CD81 for a dual mode chromatography remained comparable to the SEC step.

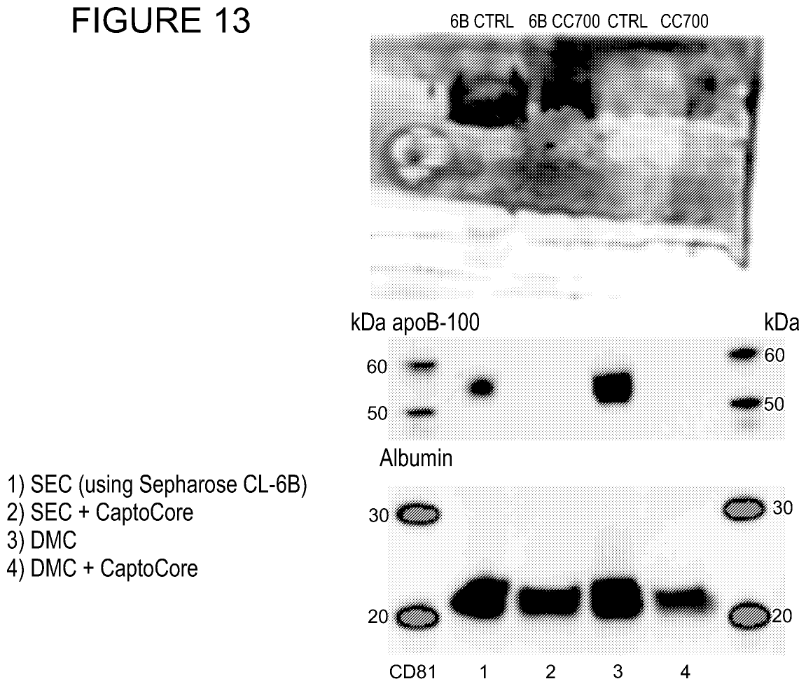

This dual mode chromatography can also be combined with Capto™ Core 700 resin to achieve an additional level of purity for EV purification. A direct comparison between fractionation of human plasma samples was demonstrated using (1) size exclusion chromatography (SEC) with a 10 mL Sepharose™ CL-6B column, (2) SEC followed by CaptoCore 700 beads, (3) dual mode chromatography (DMC) using 10 mL Sepharose™ CL-6B layered on top of 2 mL of cation exchange resin (Fractogel EMD 503), and (4) DMC followed by Capto Core 700 beads. As shown in FIG. 13, the combination of SEC and CaptoCore, or DMC and CaptoCore were able to completely remove the contaminating protein, Albumin, and the major lipoprotein component ApoB 100, while still recover a significant level of EV marker CD81. This represents a significant improvement over all existing EV purification methods.

These data demonstrate that by combining the size exclusion chromatography with another chromatography step, such as a cation exchange chromatography, a further improvement in EV purity was observed. This dual mode chromatography can also be combined with Capto™ Core 700 resin to achieve an additional level of purity for EV purification.

Comparison between Capto Core and Other Methods for EV Purification from CSF and Plasma Samples

The methods of the claimed invention were compared with other methods for EV purification. Table 3 shows a summary of the methods comparing the numbers of EV proteins being identified from CSF samples in each method. Table 4 shows a summary of the methods comparing the numbers of EV proteins being identified from plasma samples in each method. As shown in Tables 3 and 4, the methods of the present invention not only require a minimal amount of sample volume to start with, but were also able to recover the most number of EV proteins in the final product. This direct comparison clearly demonstrates that the methods of the present invention confer significant advantages over the commonly used EV purification methods.

TABLE 3
Comparison between Capto Core and Other Methods
for EV Purification from CSF Samples

| | | | Number of |
|---|---|---|---|
| | Starting | | Proteins |
| Method | volume | Methodology | Identified |
| Method of the Present | 1 mL | Capto Core | 1489 |
| Invention | | | |
| Chiasserini et al 2014 | 6 mL | Ultracentrifugation | 760 |
| Journal of Proteomics | | | |
| Thompson et al 2018 | 8 mL | Ultrafiltration Liquid | 622 |
| Proteomics | | Chromotography | |
| Muraoka et al 2019 | 3 mL | Phosphatidyl Serine | 429 |
| Frontiers in | | Magnetic Bead Capture | |
| Neuroscience | | | |

TABLE 4
Comparison between DMC and Capto Core and Other
Methods for EV Purification from Plasma Samples

| | | | | Number of |
|---|---|---|---|---|
| | Starting | | | Proteins |
| Method | volume | | Methodology | Identified |
| Method of the | 1 | mL | DMC + Capto Core | 1489 |
| Present Invention | | | | |
| Karimi et al 2018 | 40-80 | mL | Ultracentrifugation + | 871 |
| CMLS | | | Density Gradient | |
| | | | Ultracentrifugation + | |
| | | | SEC | |
| Kalra et al 2015 | 25 | mL | Ultracentrifugation + | 213 |
| Proteomics | | | Density Gradient | |
| | | | Ultracentrifugation | |
| Jhang et al 2020 | 1 | mL | PEG Precipitation + | 287 |
| JEV | | | Density Gradient | |
| | | | Ultracentrifugation + | |
| | | | SEC | |

### Discussion

The inventors have developed Simoa® assays that can be used for ultrasensitive quantitation of EVs in biological fluids. Although several technologies have been applied to EV detection, Simoa® technology has a unique set of features that makes it particularly well suited for measuring EVs, overcoming several limitations of other methods. In addition to differentiating EVs from other particles (such as lipoproteins or aggregated proteins), Simoa® also offers high dynamic range, throughput and sensitivity. Using Simoa®, the tetraspanins CD9, CD63, and CD81 can be measured at one to two orders of magnitude higher sensitivity than conventional methods such as ELISA. Critically, fractions were measured from a commercial SEC column, the majority of EV markers in CSF were not detectable using standard ELISA with the same antibodies.

Simoa®'s sensitivity is achieved by immuno-capture and counting of individual proteins in a microwell array. Simoa® has been applied to EVs, pulling down EVs into individual microwells by their transmembrane proteins. Simoa® can be used for single EV analysis, counting the number of antigen positive EV in a particular samples, or single EVs with combinations of markers by using different capture and detector antibodies.

The assays described in this Example were performed in the presence of detergents such as Tween, which may affect EVs. Although it has been previously reported that EVs remain intact in the presence of low concentrations of Tween, this was not confirmed in these studies.

Simoa was used to address one of the major questions in the EV field: what is the best method for EV purification from biological fluids? The most commonly used EV purification methods were directly compared in plasma and CSF. Although several studies have previously compared EV purification methods and yielded important insights, the lack of available quantitative methods for EV measurement has made these studies difficult to interpret. Simoa® is a framework to rapidly and directly provide a quantitative comparison of EV purification methods. By detecting three different tetraspanins per sample with Simoa®, the probability of measuring a rare subset of EVs was minimized. Accordingly, it was found there was a strong correlation of the relative levels of the three tetraspanins in different SEC fractions. By also developing a Simoa® assay to also measure the free protein albumin (which is the main contaminant of EV preps), purity of EVs were determined in addition to yield. The following EV purification methods were compared in plasma and CSF: ultracentrifugation (with or without a wash), the commonly used commercial EV precipitation reagent ExoQuick™ (as well as the newer version ExoQuick Ultra™), and SEC using the commercial Izon™ qEV columns. The results showed that there were significant differences between the performance of various methods in plasma and CSF. This could be due to the fact that plasma has a much higher protein concentration than CSF, affecting the yield and purity of a given purification method. Commercial SEC columns gave the highest yield and purity in plasma. In CSF, EV precipitation by ExoQuick™ had higher yield while SEC had higher purity.

Given the high throughput capabilities of Simoa®, several resins and column dimensions were then compared to improve EV purification using SEC. By analyzing the tetraspanin and albumin levels in each fraction, three different resins (Sepharose™ CL-2B, Sepharose™ CL-4B, and Sepharose™ CL-6B) were directly compared to the commercial Izon™ qEV SEC columns. The Sepharose™ columns yielded significant improvements in EV yield over the commercial columns at a fraction of the cost. In particular, it was found that Sepharose™ CL-6B yields considerably higher levels of EVs than Sepharose™ CL-2B or Sepharose™ CL-4B. This is likely due to the fact that Sepharose™ CL-6B beads have a smaller average pore size, leading to a lower probability that EVs will enter the beads. In SEC, it was found that there is a tradeoff between EV yield and albumin contamination.

Collecting more EVs generally means having more albumin. Thus, different SEC columns will be suited for different applications. Using a 10 mL Sepharose™ CL-6B column for EV purification from plasma or CSF is the best choice for downstream application where maximum EV yield is needed and where some free protein contamination is not detrimental. This column would be the best choice when analyzing rare EV cargo or when further purification of EVs will be performed (for example, immuno-isolation for a cell type-specific marker). On the other hand, if purification EVs from plasma where minimal free protein contamination is desired (for example, for EV protein analysis by western blot), a larger 20 mL column with Sepharose™ CL-6B yields the best results. Conversely, in CSF, larger 20 mL columns demonstrated poor yield, and therefore 10 mL columns can be used: Sepharose™ CL-6B for maximum yield or Sepharose™ CL-4B for maximum purity.

As described herein, Simoa® was used to quantify EVs after different purification methods in plasma and CSF, but the application of these methods is not limited to this application. The general experimental framework described herein can be applied to evaluate new EV purification methods in plasma or CSF, or applied to any other biological fluid, such as urine or saliva. While highly optimized Simoa® assays were developed for CD9, CD63, CD81, and albumin, other proteins can also be measured. Simoa® assays can also be developed for cell type specific EV surface proteins or for the quantification of other particles such as lipoproteins.

These methods can also be applied to the study of EVs in cell culture. The sensitivity and high-throughput nature of Simoa® allows for relative EV quantification in cell culture media from small numbers of cells. Thus, one could quantify EVs after gene editing to study EV biogenesis, or determine the effect of various cellular perturbations on EV levels. Additionally, Simoa® can be used to study EV heterogeneity of single EVs. As sensitivity of EV detection and specificity in differentiating EVs from protein aggregates and lipoproteins are obstacles in all EV studies, ultrasensitive protein detection of EV surface proteins will be applicable to both the study of basic EV biology and the development of diagnostic methods.

### REFERENCES

1. Hirshman B R, Kras R T, Akers J C, Carter B S, Chen C C. Extracellular Vesicles in Molecular Diagnostics: An Overview with a Focus on CNS Diseases. Advances in clinical chemistry. 2016; 76:37-53.
2. Tkach M, Kowal J, Thery C. Why the need and how to approach the functional diversity of extracellular vesicles. Philosophical transactions of the Royal Society of London Series B, Biological sciences. 2018; 373(1737).
3. Hartjes T A, Mytnyk S, Jenster G W, van Steijn V, van Royen M E. Extracellular Vesicle Quantification and Characterization: Common Methods and Emerging Approaches. Bioengineering (Basel, Switzerland). 2019; 6(1).
4. Shao H, Im H, Castro C M, Breakefield X, Weissleder R, Lee H. New Technologies for Analysis of Extracellular Vesicles. Chemical reviews. 2018; 118(4):1917-50.
5. Coumans F A W, Brisson A R, Buzas E I, Dignat-George F, Drees E E E, El-Andaloussi S, et al. Methodological Guidelines to Study Extracellular Vesicles. Circulation research. 2017; 120(10):1632-48.
6. Simonsen J B. What Are We Looking At? Extracellular Vesicles, Lipoproteins, or Both? Circulation research. 2017; 121(8):920-2.
7. Sodar B W, Kittel A, Paloczi K, Vukman K V, Osteikoetxea X, Szabo-Taylor K, et al. Low-density lipoprotein mimics blood plasma-derived exosomes and microvesicles during isolation and detection. Scientific reports. 2016; 6:24316.
8. Osteikoetxea X, Balogh A, Szabo-Taylor K, Nemeth A, Szabo T G, Paloczi K, et al. Improved characterization of EV preparations based on protein to lipid ratio and lipid properties. PloS one. 2015; 10(3):e0121184.
9. Visnovitz T, Osteikoetxea X, Sodar B W, Mihaly J, Lorincz P, Vukman K V, et al. An improved 96 well plate format lipid quantification assay for standardisation of experiments with extracellular vesicles. Journal of extracellular vesicles. 2019; 8(1):1565263.
10. Rissin D M, Kan C W, Campbell T G, Howes S C, Fournier D R, Song L, et al. Single-molecule enzyme-linked immunosorbent assay detects serum proteins at subfemtomolar concentrations. Nature biotechnology. 2010; 28(6):595-9.
11. Cohen L, Walt D R. Highly Sensitive and Multiplexed Protein Measurements. Chemical reviews. 2019; 119(1):293-321.
12. Coumans F A W, Gool E L, Nieuwland R. Bulk immunoassays for analysis of extracellular vesicles. Platelets. 2017; 28(3):242-8.
13. Osteikoetxea X, Sodar B, Nemeth A, Szabo-Taylor K, Paloczi K, Vukman K V, et al. Differential detergent sensitivity of extracellular vesicle subpopulations. Organic & biomolecular chemistry. 2015; 13(38):9775-82.
14. Lobb R J, Becker M, Wen S W, Wong C S, Wiegmans A P, Leimgruber A, et al. Optimized exosome isolation protocol for cell culture supernatant and human plasma. Journal of extracellular vesicles. 2015; 4:27031.
15. Helwa I, Cai J, Drewry M D, Zimmerman A, Dinkins M B, Khaled M L, et al. A Comparative Study of Serum Exosome Isolation Using Differential Ultracentrifugation and Three Commercial Reagents. PloS one. 2017; 12(1):e0170628.
16. Baranyai T, Herczeg K, Onodi Z, Voszka I, Modos K, Marton N, et al. Isolation of Exosomes from Blood Plasma: Qualitative and Quantitative Comparison of Ultracentrifugation and Size Exclusion Chromatography Methods. PloS one. 2015; 10(12):e0145686.
17. Soares Martins T, Catita J, Martins Rosa I, O ABdCES, Henriques A G. Exosome isolation from distinct biofluids using precipitation and column-based approaches. PloS one. 2018; 13(6):e0198820.
18. An M, Wu J, Zhu J, Lubman D M. Comparison of an Optimized Ultracentrifugation Method versus Size-Exclusion Chromatography for Isolation of Exosomes from Human Serum. Journal of proteome research. 2018; 17(10):3599-605.
19. Stranska R, Gysbrechts L, Wouters J, Vermeersch P, Bloch K, Dierickx D, et al. Comparison of membrane affinity-based method with size-exclusion chromatography for isolation of exosome-like vesicles from human plasma. Journal of translational medicine. 2018; 16(1):1.
20. Diaz G, Bridges C, Lucas M, Cheng Y, Schorey J S, Dobos K M, et al. Protein Digestion, Ultrafiltration, and Size Exclusion Chromatography to Optimize the Isolation of Exosomes from Human Blood Plasma and Serum. Journal of visualized experiments: JoVE. 2018(134).
21. Kalra H, Adda C G, Liem M, Ang C S, Mechler A, Simpson R J, et al. Comparative proteomics evaluation of plasma exosome isolation techniques and assessment of the stability of exosomes in normal human blood plasma. Proteomics. 2013; 13(22):3354-64.
22. Serrano-Pertierra E, Oliveira-Rodriguez M, Rivas M, Oliva P, Villafani J, Navarro A, et al. Characterization of Plasma-Derived Extracellular Vesicles Isolated by Different Methods: A Comparison Study. Bioengineering (Basel, Switzerland). 2019; 6(1).
23. Gamez-Valero A, Monguio-Tortajada M, Carreras-Planella L, Franquesa M, Beyer K, Borras F E. Size-Exclusion Chromatography-based isolation minimally alters Extracellular Vesicles' characteristics compared to precipitating agents. Scientific reports. 2016; 6:33641.
24. Takov K, Yellon D M, Davidson S M. Comparison of small extracellular vesicles isolated from plasma by ultracentrifugation or size-exclusion chromatography: yield, purity and functional potential. Journal of extracellular vesicles. 2019; 8(1):1560809.
25. Ludwig N, Whiteside T L, Reichert T E. Challenges in Exosome Isolation and Analysis in Health and Disease. International journal of molecular sciences. 2019; 20(19).
26. Kowal E J K, Ter-Ovanesyan D, Regev A, Church G M. Extracellular Vesicle Isolation and Analysis by Western Blotting. Methods Mol Biol. 2017; 1660:143-52.

What is claimed is:

1. A method for purifying extracellular vesicles from a biological sample, the method comprising:
(a) combining a liquid biological sample containing extracellular vesicles with size-exclusion beads capable of capturing molecules smaller than about 700 kDa to create a mixture, and
(b) separating and removing the size-exclusion beads from the mixture, such that the extracellular vesicles remain,
thereby purifying the extracellular vesicles.

2. The method of claim 1, wherein the biological sample is obtained from a subject.

3. The method of claim 1 or 2, wherein the liquid biological sample is a plasma sample.

4. The method of claim 1 or 2, wherein the liquid biological sample is a cerebrospinal fluid (CSF) sample.

5. The method of any of the preceding claims, wherein the size-exclusion beads are suspended in a buffer to create a slurry.

6. The method of claim 5, wherein the size-exclusion beads are suspended in an equal volume of buffer to create a slurry.

7. The method of claim 5 or 6, wherein the buffer is PBS buffer.

8. The method of any of the preceding claims, wherein the mixture is mixed prior to separating and removing the size-exclusion beads.

9. The method of claim 8, wherein the mixture is mixed for between 30 minutes and 1 hour.

10. The method of any of the preceding claims, wherein the size-exclusion beads are separated from the mixture using centrifugation.

11. The method of claim 10, wherein the centrifugation is at about 800 g.

12. The method of any of the preceding claims, wherein following separating and removing the size-exclusion beads from the mixture, the remaining extracellular vesicles are further purified.

13. The method of any of the preceding claims, wherein the size-exclusion beads comprise an inactive bead exterior.

14. The method of any of the preceding claims, wherein the interior of the beads comprise octylamine ligands.

15. The method of any of the preceding claims, wherein the size-exclusion beads comprise bind-elute resin.

16. The method of any of the preceding claims, wherein the size-exclusion beads are Capto™ Core 700 bind-elute beads.

17. The method of any of the preceding claims, wherein the size-exclusion beads are added in an amount of 20 μL, 50 μL, 100 μL, or 200 μL per 1 mL of liquid biological sample.

18. The method of any one of the preceding claims, wherein the sample is subjected to a size exclusion chromatography (SEC) column prior to combining with the size-exclusion beads capable of capturing molecules smaller than about 700 kDa.

19. The method of claim 18, wherein the size exclusion chromatography column comprises a stationary phase comprising a 6% cross-linked agarose size exclusion chromatography base matrix.

20. The method of claim 19, wherein the 6% cross-linked agarose size exclusion chromatography base matrix is a Sepharose™ CL-6B resin.

21. The method of any one of the preceding claims, wherein the sample is further subjected to a cation exchange chromatography resin after the size exclusion chromatography column and prior to combining with the size-exclusion beads capable of capturing molecules smaller than about 700 kDa.

22. The method of claim 21, wherein the cation exchange chromatography resin comprises a stationary phase comprising a functional group selected from the group consisting of sulpfhydryl, sulfonate, sulfate, carboxymethyl, sulfoethyl, sulfopropyl, phosphate and sulfonate.

23. The method of claim 21, wherein the cation exchange chromatography resin is Fractogel® EMD-SO₃⁻ resin.

24. The method of any of the preceding claims, wherein the method is a high-throughput method.

25. A method for purifying extracellular vesicles from a biological sample, the method comprising:
(a) providing a size exclusion chromatography (SEC) column comprising a stationary phase comprising a 6% cross-linked agarose size exclusion chromatography base matrix,
(b) introducing a liquid biological sample comprising extracellular vesicles into the column, and
(c) collecting fractions containing extracellular vesicles from the column,
thereby purifying the extracellular vesicles.

26. The method of claim 25, wherein the 6% cross-linked agarose size exclusion chromatography base matrix is a Sepharose™ CL-6B resin.

27. The method of claim 25 or 26, wherein the liquid biological sample is obtained from a subject.

28. The method of any one of claims 25-27, wherein the liquid biological sample is a plasma sample.

29. The method of any one of claims 25-27, wherein the liquid biological sample is a cerebrospinal fluid (CSF) sample.

30. The method of any one of claims 25-29, wherein the SEC column is a 10 mL volume column.

31. The method of any one of claims 25-29, wherein the SEC column is a 20 mL volume column.

32. The method of any one of claims 25-31, wherein fractions 6-21 are collected from the column.

33. The method of any one of claims 25-31, wherein fractions 12-27 are collected from the column.

34. The method of any one of claims 25-33, wherein the 6% cross-linked agarose size exclusion chromatography base matrix is washed in buffer prior to preparation of the column.

35. The method of claim 34, wherein the 6% cross-linked agarose size exclusion chromatography base matrix is washed multiple times in buffer prior to preparation of the column.

36. The method of claim 34, wherein the 6% cross-linked agarose size exclusion chromatography base matrix is washed four or more times in buffer prior to preparation of the column.

37. The method of any one of claims 34-36, wherein the buffer is PBS buffer.

38. The method of any one of claims 25-37, wherein the column comprising the 6% cross-linked agarose size exclusion chromatography base matrix is washed prior to contacting it with the sample.

39. The method of claim 38, wherein the column is washed with PBS prior to contacting it with the sample.

40. The method of any one of claims 25-39, wherein the fractions collected from the SEC column are further subjected to a cation exchange chromatography resin.

41. The method of claim 40, wherein the cation exchange chromatography resin comprises a stationary phase comprising a functional group selected from the group consisting of sulpfhydryl, sulfonate, sulfate, carboxymethyl, sulfoethyl, sulfopropyl, phosphate and sulfonate.

42. The method of claim 40, wherein the cation exchange chromatography resin is Fractogel® EMD-SO₃⁻ resin.

43. The method of any one of claims 25-39, wherein the fractions collected from the SEC column are further subjected to size-exclusion beads capable of capturing molecules smaller than about 700 kDa.

44. The method of any one of claims 25-39, wherein the fractions collected from the SEC column are further subjected to a cation exchange chromatography resin and size-exclusion beads capable of capturing molecules smaller than about 700 kDa.

45. A method for detecting a single antigen-antibody immunocomplex of an antigen in a biological sample containing the antigen-antibody immunocomplex, wherein the antigen is a tetraspanin from an extracellular vesicle (EV), the method comprising:
(a) contacting a biological sample containing the antigen-antibody immunocomplex with a plurality of capture objects, wherein each capture object comprises a capture probe coupled to a plurality of capture moieties that specifically bind to the antigen-antibody immunocomplex, wherein the biological sample and the plurality of capture objects are contacted at a ratio that allows for a single antigen-antibody immunocomplex to bind to a single capture object;
(b) incubating the product of step (a) for a sufficient time to allow binding of the plurality of capture objects to the antigen-antibody immunocomplex contained in the sample, thereby creating a complex of the capture object and the antigen-antibody immunocomplex;
(c) contacting the complex of the capture object and the antigen-antibody immunocomplex from step (b) with a plurality of detection probes, wherein the detection probes bind to the antigen-antibody immunocomplex within the complex of the capture object and the antigen-antibody immunocomplex from step (b);
(d) labeling the product of step (c) with a detectable moiety; and
(e) detecting the detectable moiety,
thereby detecting the single antigen-antibody immunocomplex in the sample, wherein the antigen is a tetraspanin comprised in an extracellular vesicle (EV).

46. The method of claim 45, wherein the tetraspanin antigen is selected from the group consisting of CD9, CD63, and CD81.

47. The method of any one of claims 45-46, wherein the biological sample is a liquid biological sample.

48. The method of claim 46, wherein the biological sample is a plasma sample.

49. The method of claim 46, wherein the biological sample is a cerebrospinal fluid (CSF) sample.

50. The method of any one of claims 45-49, further comprising quantifying the antigen-antibody immunocomplex in the sample by determining the number of antigen-antibody immunocomplexes bound to the capture objects.

51. The method of any one of claims 45-50, wherein the capture moiety on the capture probe is a full-length antibody, an antigen-binding fragment of an antibody, or an antibody mimetic.

52. The method of any one of claims 45-51, wherein step (e) comprises linking a detection probe to the detectable moiety by a non-covalent affinity binding pair, wherein the detection probe is linked to a first member of the non-covalent affinity binding pair, and the detectable moiety is linked to a second member of the non-covalent affinity binding pair.

53. The method of any one of claims 45-52, wherein the detection of step (e) comprises single-molecule detection of the detectable moiety.

54. The method of claim 53, wherein the detection of step (e) occurs in an array of microwells, wherein the microwells are capable of holding zero or one capture objects.

55. The method of claim 54, wherein the detectable moiety is detected in a fluid volume of about 10 attoliters (aL) to about 10 picoliters (pL) in a microwell.

56. The method of claim 54 or 55, wherein the array is a QUANTERIX™ single molecule array (Simoa).

57. The method of any one of claims 54-56, wherein the microwells have a volume of about 40 femtoliters (fL).

58. The method of any one of claims 1-44, further comprising quantifying the purified extracellular vesicles (EVs) using the method of claim 45.

59. A method for quantifying extracellular vesicles in a biological sample comprising detecting a single antigen-antibody immunocomplex of an antigen in a biological sample containing the antigen-antibody immunocomplex, wherein the antigen is a tetraspanin from an extracellular vesicle (EV), the method comprising:
(a) contacting a biological sample containing the antigen-antibody immunocomplex with a plurality of capture objects, wherein each capture object comprises a capture probe coupled to a plurality of capture moieties that specifically bind to the antigen-antibody immunocomplex, wherein the biological sample and the plurality of capture objects are contacted at a ratio that allows for a single antigen-antibody immunocomplex to bind to a single capture object;
(b) incubating the product of step (a) for a sufficient time to allow binding of the plurality of capture objects to the antigen-antibody immunocomplex contained in the sample, thereby creating a complex of the capture object and the antigen-antibody immunocomplex;
(c) contacting the complex of the capture object and the antigen-antibody immunocomplex from step (b) with a plurality of detection probes, wherein the detection probes bind to the antigen-antibody immunocomplex within the complex of the capture object and the antigen-antibody immunocomplex from step (b);
(d) labeling the product of step (c) with a detectable moiety; and
(e) detecting the detectable moiety,
thereby detecting the single antigen-antibody immunocomplex in the sample, and detecting the EVs in the sample.
